(19) United States
(12) Patent Application Publication
(10) Pub. No.: US 2024/0290215 A1
(43) Pub. Date: Aug. 29, 2024

(54) INFORMATION PROCESSING DEVICE, INFORMATION PROCESSING SYSTEM, INFORMATION PROCESSING METHOD, AND COMPUTER PROGRAM

(71) Applicant: National University Corporation Tokai National Higher Education and Research System, Nagoya-shi, Aichi (JP)

(72) Inventors: Kan SHIMAZAKI, Nagoya-shi, Aichi (JP); Hirofumi AOKI, Nagoya-shi, Aichi (JP); Hiroko SHINKAI, Nagoya-shi, Aichi (JP); Makoto INAGAMI, Nagoya-shi, Aichi (JP)

(21) Appl. No.: 18/696,011

(22) PCT Filed: Sep. 29, 2022
(86) PCT No.: PCT/JP2022/036387

§ 371 Date: Mar. 27, 2024

(30) Foreign Application Priority Data
Sep. 30, 2021 (JP) 2021-160624

(51) Int. Cl. G09B 9/05; A61B 5/18; G06F 3/01

(52) U.S. Cl. CPC G09B 9/05; A61B 5/18; G06F 3/011

(57) ABSTRACT

An information processing device includes a speed selection instruction receiving unit, a display control unit, a location-pointing action accepting unit, and a characteristic measurement unit. The speed selection instruction receiving unit receives a speed selection instruction from the examinee. The display control unit executes a speed-variable display process to display a simulated moving image, which is a moving image simulating the field of vision of a moving human and including at least one target object in such a manner that the moving speed is set in accordance with the speed selection instruction. The location-pointing action accepting unit accepts a location-pointing action performed by the examinee to point to a specific location in the simulated moving image. The characteristic measurement unit measures the characteristics based on the indication results correlating with whether the location-pointing action correctly point to the location of the target object.

### TECHNICAL FIELD

The technology disclosed herein relates to an information processing device that measures the characteristics of an examinee when the examinee moves.

### BACKGROUND ART

It is useful to appropriately measure and evaluate the characteristics (e.g., target object perception ability for hazards or the like, self-evaluation ability, self-control ability, risk-taking tendency, risk level, and the like) of a driver when the driver is driving a car and moving on a road. For example, when issuing or renewing a driver's license, measuring such driving characteristics to make a decision on whether to issue or renew a driver's license based on the results of such characteristic measurement, or to provide training or drills based on the results of such characteristic measurement, can reduce the average risk of drivers, which will lead to the prevention of traffic accidents. In particular, since the elderly may have impaired cognitive functions, and the elderly or those who have developed eye diseases, such as glaucoma, may have a narrowed or deficient field of vision, which tends to increase their risk while driving, it is extremely useful to measure the driving characteristics of such persons.

A method has been proposed for evaluating risk, which is one of the characteristics of a driver while driving, in which a moving image simulating the driver's field of vision is displayed in front of the examinee using an image display device (LCD monitor or projector), and when the examinee recognizes a hazard (car, bicycle, and pedestrian, among others) in the moving image, the examinee responds verbally or by pressing a button, and the like, thereby evaluating the examinee's driving risk based on the results of the test (see, e.g., Non-Patent Literature 1).

### CITATION LIST

### Non Patent Literature

Non-Patent Literature 1: “An Analysis of Elderly Drivers' Hazard Perception on Expressway” Masahiro TADA, and 5 others, JSTE Journal of Traffic Engineering, Volume 2, Issue 2, Pages A_75-A_84, Published: Feb. 5, 2016

### SUMMARY OF INVENTION

### Technical Problem

While actually driving a car, the increased risk caused by cognitive function decline and visual field narrowing or loss can be compensated for by the action of reducing the driving speed at which the car is driven. In the conventional technology described above, since the examinee cannot perform the operation (instruction) to reduce the driving speed of the car during the test, risk evaluation considering such compensatory action cannot be performed, and there is room for improvement in terms of the appropriateness of the characteristic measurement for drivers.

Such issues are not limited to the cases of measuring the characteristics of drivers while driving a car, but are also common to the cases of measuring the characteristics of drivers while driving other types of vehicles (e.g., bicycles) and pedestrians while walking.

This specification discloses technologies that can solve the above-mentioned issues.

### Solution to Problem

The technology disclosed herein can be implemented e.g., in the following aspects.

(1) The information processing device disclosed herein is an information processing device for measuring characteristics of the examinee when the examinee moves, and includes a speed selection instruction receiving unit, a display control unit, a location-pointing action accepting unit, and a characteristic measurement unit. The speed selection instruction receiving unit receives a speed selection instruction for selecting a moving speed by the examinee. The display control unit executes a speed-variable display process to display on an image display unit a simulated moving image, which is a moving image simulating the field of vision of a human moving along a predetermined course and including at least one target object in such a manner that the moving speed is set in accordance with the speed selection instruction. The location-pointing action accepting unit accepts a location-pointing action performed by the examinee to point to a specific location in the simulated moving image. The characteristic measurement unit measures the characteristics based on the indication results correlating with whether the location-pointing action correctly pointed to the location of the target object at a specific time when the scene including each target object in the simulated moving image is displayed in the speed-variable display process and on the content of the speed selection instruction, and outputs characteristic information representing the measurement results of the characteristics.

Thus, in this information processing device, the display control unit executes the speed-variable display process in which the simulated moving image is displayed in such a manner that the moving speed is set according to the speed selection instruction received by the speed selection instruction receiving unit, and the characteristic measurement unit measures the characteristics of the examinee based on the content of the speed selection instruction in addition to the indication results correlating with whether the location-pointing action correctly pointed to the location of the target object at a specific time when the scene including each target object in the simulated moving image is displayed in the speed-variable display process. Therefore, this information processing device can perform the characteristic measurement for the examinee while considering the action of the examinee to select the moving speed, thereby improving the appropriateness of the characteristic measurement for the examinee.

(2) In the above information processing device, the display control unit may be configured to execute a speed-fixed display process to display the simulated moving image on the image display unit in a manner in which the moving speed is fixed to a preset reference speed, and the characteristic measurement unit may be configured to measure the characteristics based on the indication results in the speed-fixed display process. This information processing device can measure the characteristics of the examinee by comparing the indication results in each of the speed-fixed display process, in which the moving speed is fixed to a reference speed, and the speed-variable display process, in which the examinee can select the moving speed, thereby effectively improving the appropriateness of the characteristic measurement for the examinee.

(3) In the above information processing device, the display control unit may be configured to execute the speed-variable display process after execution of the speed-fixed display process, and the speed selection instruction receiving unit may be configured to receive the speed selection instruction after execution of the speed-fixed display process and before execution of the speed-variable display process. This information processing device allows the examinee to select the moving speed in the speed-variable display process based on the feeling of his/her own indication result in the speed-fixed display process, thereby accurately grasping the self-evaluation ability and the self-control ability of the examinee and effectively improving the appropriateness of the characteristic measurement for the examinee.

(4) In the above information processing device, the characteristics may include the target object perception ability and the self-control ability of the examinee. This information processing device can effectively improve the appropriateness of the characteristic measurement for the examinee including the target object perception ability and the self-control ability of the examinee.

(5) In the above information processing device, the characteristics may include the self-evaluation ability of the examinee, and the information processing device may further include a self-evaluation receiving unit that receives a self-evaluation of the indication results evaluated by the examinee, and the characteristic measurement unit may measure the characteristics based on the self-evaluation. This information processing device can measure the characteristics of the examinee based on the self-evaluation by the examinee of the indication results, thereby effectively improving the appropriateness of the characteristic measurement for the examinee, including the self-evaluation ability of the examinee.

(6) In the above information processing device, the characteristic measurement unit may be configured to determine the self-evaluation ability of the examinee based on the difference between the self-evaluation and the actual indication results. This information processing device can further effectively improve the appropriateness of the characteristic measurement for the examinee, including the self-evaluation ability of the examinee.

(7) In the above information processing device, the speed selection instruction receiving unit may be configured to receive the speed selection instruction during the speed-variable display process, and the display control unit may be configured to change the speed of movement according to the speed selection instruction during the speed-variable display process. This information processing device can perform the characteristic measurement for the examinee while considering the actions closer to the actual moving environment, such as changing (accelerating or decelerating) the moving speed while moving, thereby effectively improving the appropriateness of the characteristic measurement for the examinee.

(8) In the above information processing device, the characteristic may include a risk of the examinee while moving, and the characteristic measurement unit may be configured to determine that in a case where there is no location-pointing action correctly pointing to the location of the target object at the specific time during the speed-variable display process, the risk is lower when there is a speed selection instruction to reduce the moving speed than when there is no speed selection instruction to reduce the moving speed. In a case where the examinee failed to recognize the target object at the specific time during the speed-variable display process, when there is a speed selection instruction to reduce the speed of movement (hereinafter also referred to as the “deceleration instruction”), it is assumed that the examinee took compensatory action due to metacognition of his/her own cognitive deficiency, while when there is no deceleration instruction, it is assumed that the examinee did not take compensatory action due to a lack of metacognition of his/her own cognitive deficiency. This information processing device can determine the risk when such a compensatory action is taken to be lower than when no compensatory action is taken, thereby improving the appropriateness of the risk measurement as the characteristic measurement for the examinee.

(9) In the above information processing device, the characteristics may include a risk of the examinee while moving, and the characteristic measurement unit may be configured to determine that in a case where there is no location-pointing action correctly pointing to the location of the target object at the specific time during the speed-fixed display process and there is no location-pointing action correctly pointing to the location of the target object at the specific time during the speed-variable display process, the risk is lower when there is a speed selection instruction to reduce the moving speed than when there is no speed selection instruction to reduce the moving speed. In a case where the examinee failed to recognize the target object at the specific time during both the speed-fixed display process and the speed-variable display process, when there is a deceleration instruction, it is assumed that the examinee took compensatory action due to metacognition of his/her own cognitive deficiency, while when there is no deceleration instruction, it is assumed that the examinee did not take compensatory action due to a lack of metacognition of his/her own cognitive deficiency. This information processing device can determine the risk when such a compensatory action is taken to be lower than when no compensatory action is taken, thereby improving the appropriateness of the risk measurement as the characteristic measurement for the examinee.

(10) In the above information processing device, the characteristic measurement unit may be configured to determine that in a case where there is no location-pointing action correctly pointing to the location of the target object at the specific time during the speed-fixed display process and there is a location-pointing action correctly pointing to the location of the target object at the specific time during the speed-variable display process, the risk is lower when there is a speed selection instruction to reduce the moving speed than when there is no speed selection instruction to reduce the moving speed. According to this information processing device, even in cases where the examinee could recognize the target object during the speed-variable display process but failed to recognize the target object during the speed-fixed display process, when there is a deceleration instruction, it is assumed that the examinee took compensatory action due to metacognition of his/her own cognitive deficiency, while when there is no deceleration instruction, it is assumed that the examinee did not take compensatory action due to a lack of metacognition of his/her own cognitive deficiency. This information processing device can determine the risk when such a compensatory action is taken to be lower than when no compensatory action is taken, thereby improving the appropriateness of the risk measurement as the characteristic measurement for the examinee.

(11) In the above information processing device, the display control unit may be configured to execute the speed-variable display process after execution of the speed-fixed display process. This information processing device can accurately grasp the level of the metacognitive ability of the examinee based on the presence or absence of the speed selection instruction to decrease the speed of movement in the subsequent speed-variable display process for a scene containing a target object that could not be recognized in the speed-fixed display process, thereby effectively improving the appropriateness of the characteristic measurement for the examinee.

(12) In the above information processing device, the simulated moving image may include the target object representing actual risk (such as visible cars, bicycles, and pedestrians) and the target object representing potential risk (such as blind spots), the characteristics may include the risk of the examinee while moving, and the characteristic measurement unit may be configured to measure the risk due to a visual field defect based on the indication result at the specific time when a scene including the target object representing actual risk is displayed, and the risk due to cognitive function decline based on the indication result at the specific time when a scene including the target object representing potential risk is displayed. This information processing device can improve the appropriateness of the risk measurement for risks caused by the visual field defect and the appropriateness of the risk measurement for risks caused by the cognitive function decline.

(13) In the above information processing device, the simulated moving image may be configured to be a moving image simulating the field of vision of a human driving a vehicle and moving on the course. This information processing device can improve the appropriateness of the characteristic measurement for the examinee when the examinee drives the vehicle and moves.

(14) The above information processing device may be configured to further include the image display unit. This information processing device can simplify the device configuration for performing the characteristic measurement.

(15) The information processing system may be configured to include the information processing device according to any one of claims 1 to 8 and an image display device as the image display unit. This information processing system can provide a system capable of appropriately measuring the characteristics of an examinee when the examinee moves while allowing the examinee to view a simulated moving image.

The techniques disclosed herein can be implemented in various forms, such as information processing devices, information processing systems, information processing methods, computer programs that implement those methods, non-transitory recording media that record those computer programs, and the like.

### BRIEF DESCRIPTION OF DRAWINGS

FIG. 1 is an explanatory diagram illustrating a schematic configuration of an information processing device 300 in the first embodiment.

FIG. 2 is a block diagram schematically illustrating a configuration of the information processing device 300 in the first embodiment.

FIG. 3 is a flowchart illustrating the contents of an examinee-characteristic measurement process in the first embodiment.

FIG. 4 is an explanatory diagram illustrating an example of a state in which a user interface is displayed on a display unit 352 during the examinee-characteristic measurement process in the first embodiment.

FIG. 5 is an explanatory diagram illustrating an example of a state in which a user interface is displayed on a display unit 352 during the examinee-characteristic measurement process in the first embodiment.

FIG. 6 is an explanatory diagram illustrating an example of a method of measuring characteristics in the examinee-characteristic measurement process in the first embodiment.

FIG. 7 is an explanatory diagram illustrating an example of a method of measuring characteristics in the examinee-characteristic measurement process in the first embodiment.

FIG. 8 is an explanatory diagram illustrating a schematic configuration of an information processing device 300 in the second embodiment.

FIG. 9 is a flowchart illustrating the contents of an examinee-characteristic measurement process in the second embodiment.

FIG. 10 is an explanatory diagram illustrating an example of a state in which characteristic information CHI indicating the result of the examinee-characteristic measurement process in the second embodiment is displayed on a display unit 352.

FIG. 11 is a flowchart illustrating the contents of an examinee-characteristic measurement process in the third embodiment.

FIG. 12 is an explanatory diagram illustrating an example of a state in which characteristic information CHI indicating the result of the examinee-characteristic measurement process in the third embodiment is displayed on the display unit 352.

FIG. 13 is an explanatory diagram illustrating a schematic configuration of an information processing system 10 in the fourth embodiment.

FIG. 14 is a block diagram illustrating a schematic configuration of an information processing system 10 in the fourth embodiment.

FIG. 15 is a flowchart illustrating the contents of an examinee-characteristic measurement process in the fourth embodiment.

FIG. 16 is an explanatory diagram schematically illustrating a state of the examinee EX during the examinee-characteristic measurement process in the fourth embodiment and the simulated driving image SI viewed by the examinee EX.

FIG. 17 is an explanatory diagram schematically illustrating a state of the examinee EX during the examinee-characteristic measurement process in the fourth embodiment and the simulated driving image SI viewed by the examinee EX.

FIG. 18 is an explanatory diagram illustrating an example of a state in which characteristic information CHI indicating the result of the examinee-characteristic measurement process in the fourth embodiment is displayed on the display unit 152.

### DESCRIPTION OF EMBODIMENTS

### A. First Embodiment

It is useful to appropriately measure the characteristics of a driver when the driver is driving a car and moving on a road. For example, when issuing or renewing a driver's license, measuring such driving characteristics to make a decision on whether to issue or renew a driver's license based on the results of such characteristic measurement, or to provide training or drills based on the results of such characteristic measurement, can reduce the average risk of drivers, which will lead to the prevention of traffic accidents. In particular, the elderly may have impaired cognitive functions, and the elderly or those who have developed eye diseases, such as glaucoma, may have a narrowed or deficient field of vision, which tends to increase their risk while driving. With regard to this, while actually driving a car, the increased risk caused by cognitive function decline and visual field narrowing or loss can be compensated for by the action of reducing the driving speed at which the car is driven. Therefore, it is extremely useful to measure driving characteristics while considering such compensatory actions. Examples of using the technology disclosed herein to appropriately measure driving characteristics of a driver (an example applied to an information processing device 300) is described below.

### A-1. Configuration of Information Processing Device 300

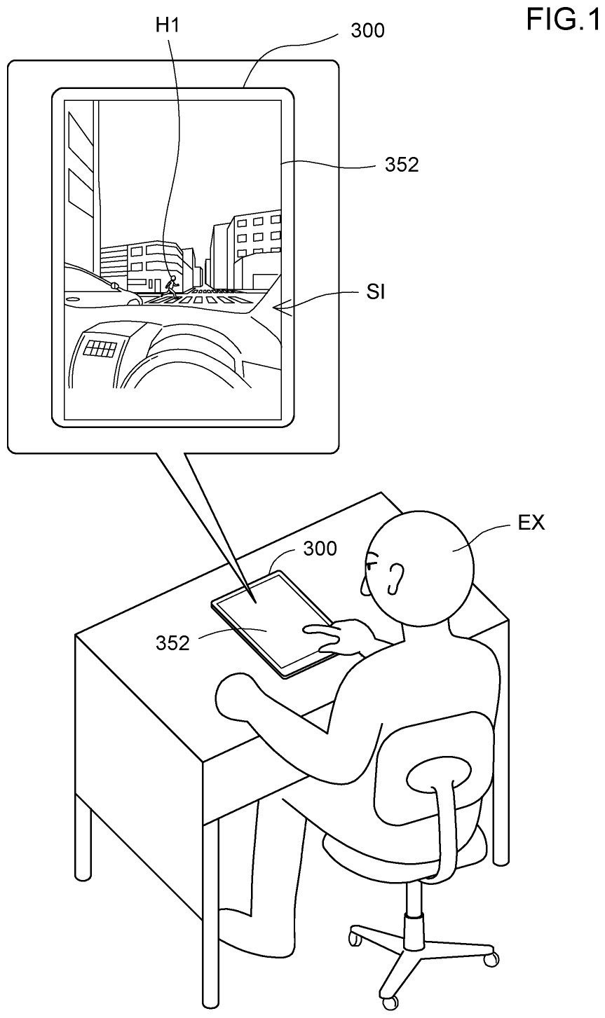

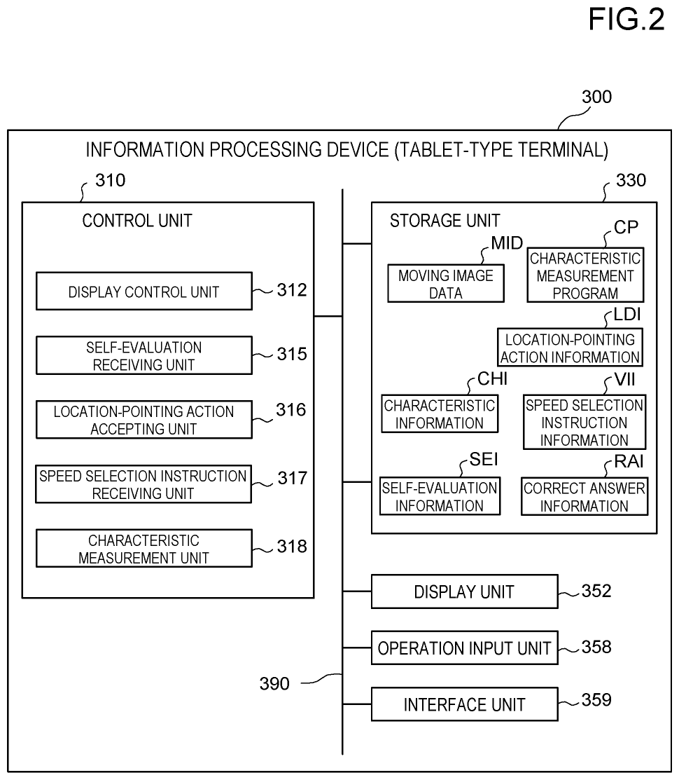

FIG. 1 is an explanatory diagram illustrating a schematic configuration of an information processing device 300 in the first embodiment and FIG. 2 is a block diagram schematically illustrating a configuration of the information processing device 300 in the first embodiment. The information processing device 300 in this embodiment is a device for measuring the characteristics of an examinee EX when the examinee EX drives a car and moves along a road. In particular, the information processing device 300 is a device that causes the examinee EX to view a simulated driving image SI that simulates the field of vision of a human driving a car and moving along a predetermined course and performs a hazard recognition test to determine whether the examinee EX recognized each hazard (dangerous point in the moving environment) included in the simulated driving image SI to measure the characteristics of the examinee EX when he/she drives a car and moves on a road based on the results of the hazard recognition test.

As shown in FIGS. 1 and 2, a tablet-type terminal is used as the information processing device 300 in this embodiment. The information processing device 300 has a control unit 310, a storage unit 330, a display unit 352, an operation input unit 358, and an interface unit 359. These units are communicatively connected to each other via a bus 390.

The display unit 352 of the information processing device 300 is composed of, e.g., a liquid crystal display, an organic EL display, or the like, and displays various images and information. The display unit 352 is an example of the image display unit in the claims. The operation input unit 358 is composed of, e.g., buttons, a microphone, or the like, and accepts operations and instructions from the administrator or the examinee EX. In this embodiment, the display unit 352 has a touch panel, which also functions as the operation input unit 358. The interface unit 359 is composed of, e.g., a LAN interface, USB interface, or the like, and enables communication with other devices by wired or wireless means.

The storage unit 330 of the information processing device 300 is composed of, e.g., ROM, RAM, a hard disk drive (HDD), or the like and is used to store various programs and data or as a work area or temporary storage area for data when executing various programs. For example, the storage unit 330 stores a characteristic measurement program CP, which is a computer program for executing the examinee-characteristic measurement process described below. The characteristic measurement program CP is provided, e.g., as stored in a computer-readable recording medium (not shown) such as a CD-ROM, DVD-ROM, USB memory, or the like and is installed in the information processing device 300 to be stored in the storage unit 330. The characteristic measurement program CP need not be stored in the storage unit 330 of the information processing device 300, but the characteristic measurement program CP may be stored in an external server, and the information processing device 300 may access the characteristic measurement program CP via a network to implement the functions of the characteristic measurement program CP on the information processing device 300.

The storage unit 330 of the information processing device 300 stores moving image data MID. The moving image data MID is data representing the simulated driving image SI described above. The simulated driving image SI is a moving image composed at a predetermined frame rate (e.g., 70 fps). The simulated driving image SI is a moving image that simulates the field of vision of a human driving a car and moving along a predetermined course, and includes a plurality of scenes including hazards Hn (n=1, 2, . . . ). The hazards Hn include, e.g., at least one of the actual hazards such as cars, bicycles, pedestrians, or the like, and potential hazards, such as intersections with poor visibility, blind spots of buildings and parked vehicles, or the like. For example, in one scene in the simulated driving image SI, a pedestrian as hazard H1 jumps out from the side of the road, as shown in FIG. 1. In this specification, a scene in a simulated driving image SI may be an image represented by a single frame constituting the simulated driving image SI or may be an image having a predetermined temporal length represented by multiple consecutive frames. The number of hazards Hn included in a scene may be one or more. The simulated driving image SI corresponds to the simulated moving image in the claims.

In this embodiment, the moving image data MID is data that enables the displayed simulated driving image SI to be changed so that the driving speed of the car is set according to the speed selection instruction from the examinee EX as described below. The speed selection instruction is at least one of a deceleration instruction to decrease the speed, an acceleration instruction to increase the speed, and a speed maintenance instruction to maintain the current speed. The moving image data MID representing the simulated driving image SI may be generated, e.g., by computer graphics (CG) software or by using images captured by a camera while driving a car on a real road. The moving image data MID may also include audio data representing a sound that imitates the sound of a car running or the like.

The storage unit 330 of the information processing device 300 also stores correct answer information RAI. The correct answer information RAI is information that identifies the timing at which the scene containing each hazard Hn is displayed in the simulated driving image SI (display time of the frame containing each hazard Hn) and the location of each hazard Hn (coordinates of the image area representing the hazard Hn on the frame). The storage unit 330 of the information processing device 300 stores location-pointing action information LDI, speed selection instruction information VII, characteristic information CHI, and self-evaluation information SEI during the examinee-characteristic measurement process described below. The contents of these pieces of information are explained in conjunction with the description of the examinee-characteristic measurement process described below.

The control unit 310 of the information processing device 300 is composed of, e.g., a CPU or the like, and controls the operation of the information processing device 300 by executing a computer program loaded from the storage unit 330. For example, the control unit 310 executes the examinee-characteristic measurement process described below by loading and executing the characteristic measurement program CP from the storage unit 330. More specifically, the control unit 310 functions as the display control unit 312, the self-evaluation receiving unit 315, the location-pointing action accepting unit 316, the speed selection instruction receiving unit 317, and the characteristic measurement unit 318 to execute the examinee-characteristic measurement process described below. The functions of each of these units are explained in conjunction with the description of the examinee-characteristic measurement process described below.

### A-2. Examinee-Characteristic Measurement Process

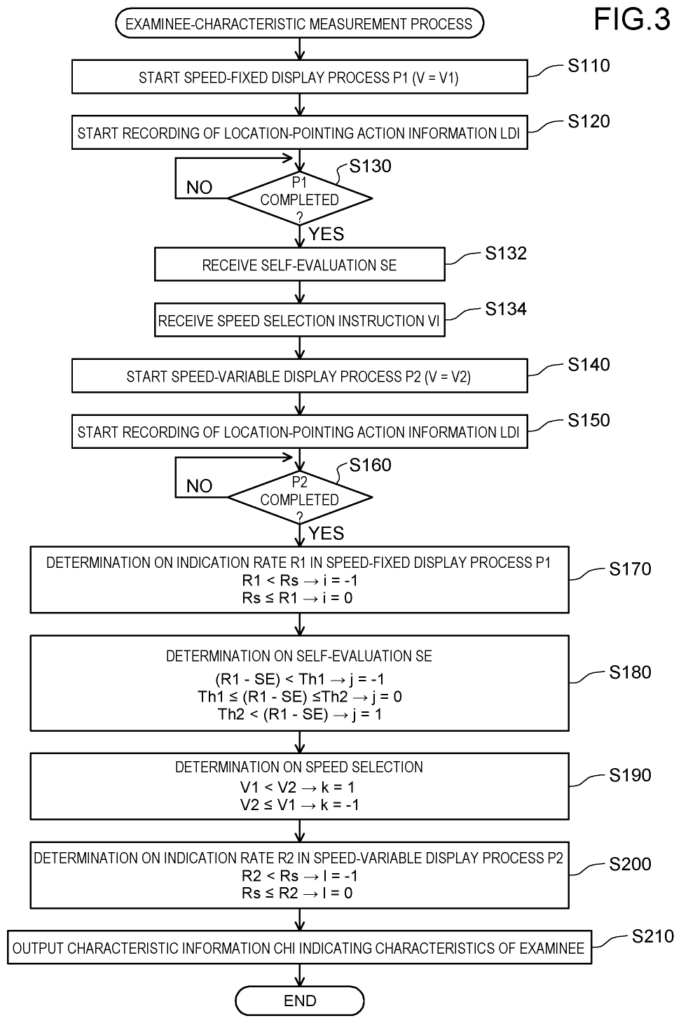

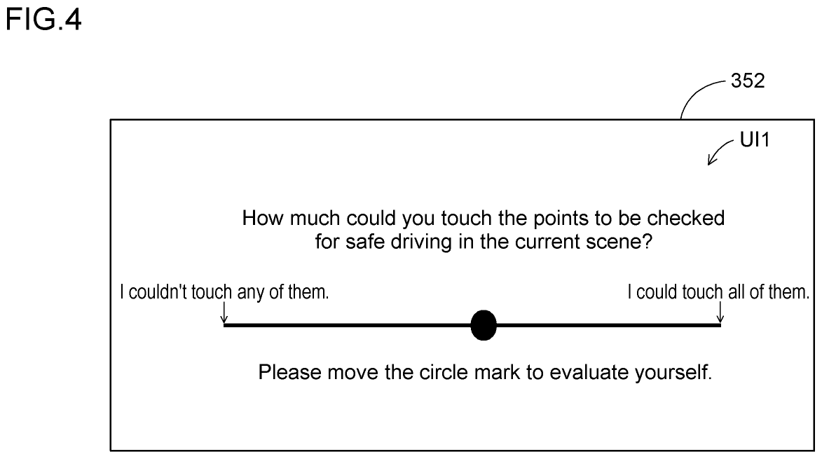

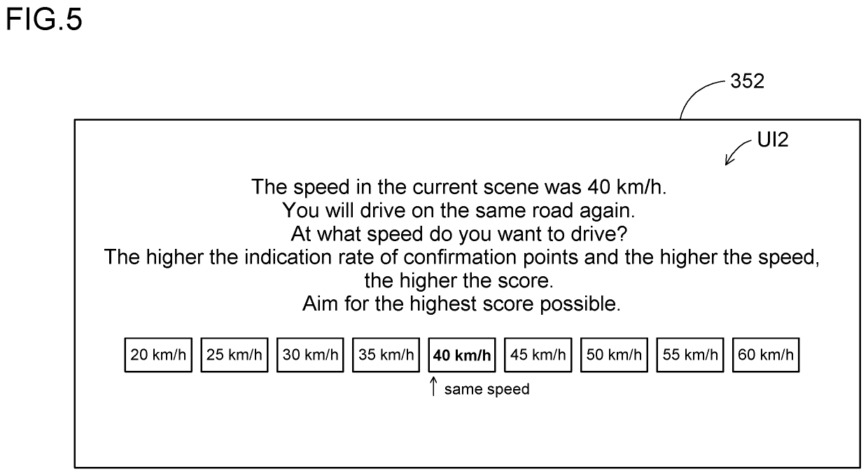

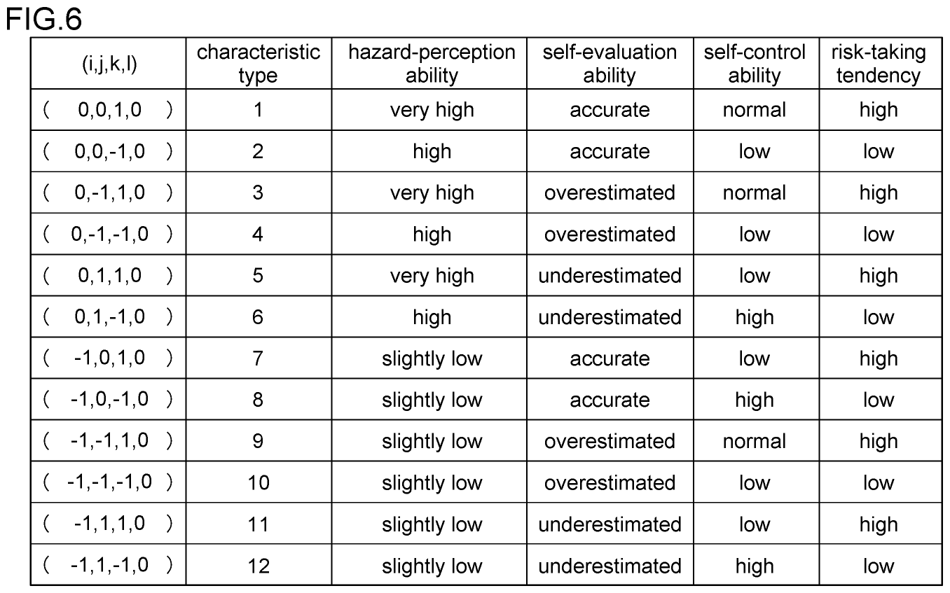

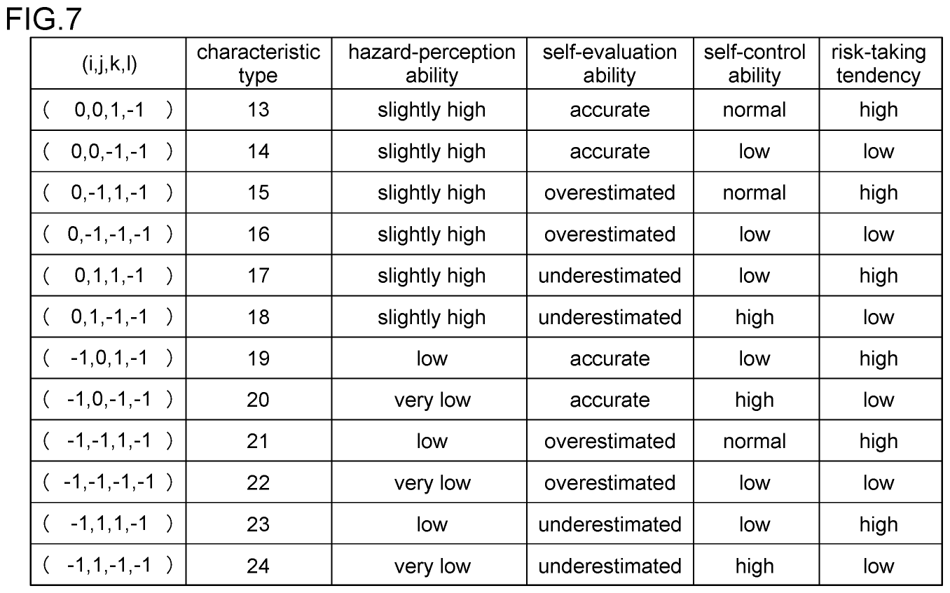

Next, the examinee-characteristic measurement process executed by the information processing device 300 of this embodiment will be described. FIG. 3 is a flowchart illustrating the contents of an examinee-characteristic measurement process in the first embodiment. FIGS. 4 and 5 are explanatory diagrams illustrating an example of a state in which a user interface (UI) is displayed on a display unit 352 during the examinee-characteristic measurement process in the first embodiment, and FIGS. 6 and 7 are explanatory diagrams illustrating an example of a method of measuring characteristics in the examinee-characteristic measurement process in the first embodiment.

The examinee-characteristic measurement process is a process that causes the examinee EX to view a simulated driving image SI and performs a hazard recognition test to determine whether the examinee EX correctly recognized each hazard Hn in the simulated driving image SI to measure the characteristics of the examinee EX when the examinee EX drives a car and moves on a road based on the results of the hazard recognition test. In the hazard recognition test, while the simulated driving image SI is displayed on the display unit 352, the examinee EX is instructed to touch the location on the display unit 352 where a hazard Hn is displayed if the examinee EX recognizes something that he/she considers to be the hazard Hn. The examinee-characteristic measurement process is started, e.g., in response to the examinee EX or the administrator inputting an instruction to start the process via the operation input unit 358 of the information processing device 300.

When the examinee-characteristic measurement process is started, the display control unit 312 of the information processing device 300 causes the display unit 352 to start displaying (reproducing) the simulated driving image SI (S110) based on the moving image data MID stored in the storage unit 330. This display process of the simulated driving image SI is hereinafter referred to as “speed-fixed display process P1”. In the speed-fixed display process P1, the display control unit 312 displays the simulated driving image SI in a manner in which the driving speed V is fixed to a preset reference speed V1 (e.g., 40 km/h). In other words, in the speed-fixed display process P1, the examinee EX cannot change the driving speed by the speed selection instruction.

Simultaneously with the start of the speed-fixed display process P1, the location-pointing action accepting unit 316 of the information processing device 300 starts accepting location-pointing actions by the examinee EX to point to a specific location in the simulated driving image SI and starts recording the location-pointing action information LDI indicating the history of such location-pointing actions (S120). The location-pointing action information LDI is information indicating the location (coordinates) on the simulated driving image SI specified by the examinee EX by touching the display unit 352 and the time when the touch operation was performed (time point in the simulated driving image SI). By referring to the location-pointing action information LDI, it is possible to know in which scene and at which location in the simulated driving image SI the examinee EX recognized the hazard Hn. The received and updated location-pointing action information LDI is stored in the storage unit 330. When a location-pointing action (touch operation to the display unit 352) is performed by the examinee EX, a response process (e.g., output of sound, some display in the display unit 352, or the like) indicating that the location-pointing action has been accepted may be performed by the control unit 310.

The display control unit 312 of the information processing device 300 monitors whether the speed-fixed display process P1 is completed (S130), and if it is determined that the speed-fixed display process P1 is completed (S130: YES), the process proceeds to S132.

Next, the self-evaluation receiving unit 315 of the information processing device 300 receives a self-evaluation SE of the indication rate evaluated by the examinee EX (S132). Here, the indication rate means the ratio of the number of hazards Hn correctly recognized by the examinee EX to the number of hazards Hn included in the simulated driving image SI in the hazard recognition test. The indication rate is an index value that correlates to whether there was a locational indication that correctly specifies the location of a hazard Hn at a specific time when a scene including each hazard Hn in the simulated driving image SI is displayed, and is an example of the indication result in the claims.

The self-evaluation receiving unit 315 displays a user interface UI1 on the display unit 352 for receiving a self-evaluation SE of the indication rate evaluated by the examinee EX, e.g., as shown in FIG. 4. This user interface UI1 includes a message prompting the examinee EX for the self-evaluation SE of the indication rate (e.g., “How much could you touch the points to be checked for safe driving in the current scene?”) and an interface for the examinee EX to input a self-evaluation SE of the indication rate (e.g., a straight line indicating each step from 0% (left end) to 100% (right end) of the indication rate, and a pointer that the examinee EX can move along the straight line to indicate one point on the straight line). The examinee EX moves the pointer to the location associated with the self-evaluation SE of the indication rate by touching the display unit 352. The self-evaluation receiving unit 315 receives the self-evaluation SE of the indication rate evaluated by the examinee EX based on the location of the pointer. The received self-evaluation SE is stored in the storage unit 330 as self-evaluation information SEI indicating the self-evaluation SE.

Next, the speed selection instruction receiving unit 317 of the information processing device 300 receives a speed selection instruction VI issued by the examinee EX to select a driving speed (S134). For example, as shown in FIG. 5, the speed selection instruction receiving unit 317 displays a user interface UI2 on the display unit 352 for receiving a speed selection instruction VI issued by the examinee EX to select a driving speed. This user interface UI2 includes a message prompting the examinee EX for the speed selection instruction (e.g., “The speed in the current scene was 40 km/h. You will drive on the same road again. At what speed do you want to drive? The higher the indication rate of confirmation points and the higher the speed, the higher the score. Aim for the highest score possible.”) and an interface for the examinee EX to select the driving speed (e.g., an icon showing nine speed choices inscribed at 5 km intervals from 20 km/h to 60 km/h). The examinee EX inputs the speed selection instruction VI by selecting the icon corresponding to the desired driving speed by touching the display unit 352. The speed selection instruction receiving unit 317 receives the speed selection instruction VI by the examinee EX based on the selected icon. The received speed selection instruction VI is stored in the storage unit 330 as speed selection instruction information VII indicating the speed selection instruction VI. Hereinafter, the driving speed selected by speed selection instruction VI is referred to as selected speed V2. The above example of a message prompting the speed selection instruction is useful for grasping the actual characteristics of the examinee EX, as it is intended to help the examinee EX avoid the behavior of trying to increase the indication rate by selecting an excessively low driving speed that is not based on his/her own ability or characteristics.

Next, the display control unit 312 of the information processing device 300 causes the display unit 352 to start displaying the simulated driving image SI based on the moving image data MID again (S140). This display process of the simulated driving image SI is hereinafter referred to as “speed-variable display process P2”. In the speed-variable display process P2, the display control unit 312 causes the simulated driving image SI to be displayed in such a manner that the driving speed V is set according to the speed selection instruction VI issued by the examinee EX. In other words, the display control unit 312 causes the display unit 352 to display the simulated driving image SI so that the driving speed V becomes the selected speed V2 selected by the speed selection instruction VI. Once the display of the simulated driving image SI in the speed-variable display process P2 has started, the driving speed V is fixed to the selected speed V2. Thus, in this embodiment, since the examinee EX does not need to perform the operation for the speed selection instruction VI while viewing the simulated driving image SI, the burden on the examinee EX is reduced, and the hazard-perception ability of the examinee EX can be measured more accurately.

When the speed-variable display process P2 is started, as in the speed-fixed display process P1 described above, the location-pointing action accepting unit 316 of the information processing device 300 starts accepting location-pointing actions by the examinee EX to point to a specific location in the simulated driving image SI and starts recording the location-pointing action information LDI indicating the history of such location-pointing actions (S150). The received and updated location-pointing action information LDI is stored in the storage unit 330.

The display control unit 312 of the information processing device 300 monitors whether the speed-variable display process P2 is completed (S160), and if it is determined that the speed-variable display process P2 is completed (S160: YES), the process proceeds to S170.

When the speed-fixed display process P1 and the speed-variable display process P2 are completed, the characteristic measurement unit 318 of the information processing device 300 starts the characteristic measurement for the examinee EX during driving with reference to the correct answer information RAI stored in advance in the storage unit 330, the location-pointing action information LDI generated and updated during execution of the speed-fixed display process P1 and the speed-variable display process P2, and the self-evaluation information SEI and the speed selection instruction information VII that were received during the speed-fixed display process P1 and the speed-variable display process P2 and stored in the storage unit 330, as described in detail below.

The characteristic measurement unit 318 of the information processing device 300 makes a determination on the indication rate R1 in the speed-fixed display process P1 (S170). Specifically, the characteristic measurement unit 318 calculates the indication rate R1 in the speed-fixed display process P1 and compares the indication rate R1 with a reference indication rate Rs. The reference indication rate Rs is set in advance as a value of the indication rate required for safe operation and stored in the storage unit 330. The characteristic measurement unit 318 sets the value of the first coefficient i to “−1” when the indication rate R1 in the speed-fixed display process P1 is below the reference indication rate Rs (i.e., when the indication rate R1 is relatively low) and sets the value of the first coefficient i to “0” when the indication rate R1 is above the reference indication rate Rs (i.e., when the indication rate R1 is relatively high).

The characteristic measurement unit 318 of the information processing device 300 makes a determination on the self-evaluation SE of the indication rate in the speed-fixed display process P1 (S180). Specifically, the characteristic measurement unit 318 calculates the difference (R1−SE) between the actual indication rate R1 in the speed-fixed display process P1 and the self-evaluation SE of the indication rate by the examinee EX identified by the self-evaluation information SEI stored in the storage unit 330, and compares the difference (R1−SE) with the first threshold Th1 and the second threshold Th2 (note that Th1<Th2). The characteristic measurement unit 318 sets the value of the second coefficient j to “−1” when the difference (R1−SE) is below the first threshold Th1 (i.e., the self-evaluation SE of the indication rate is excessive relative to the actual indication rate R1), sets the value of the second coefficient j to “0”, when the difference (R1−SE) is the first threshold Th1 or more and the second threshold Th2 or less (i.e., the self-evaluation SE of the indication rate is close to the actual indication rate R1), and sets the value of the second coefficient j to “1” when the difference (R1−SE) exceeds the second threshold Th2 (i.e., when the self-evaluation SE of the indication rate is underestimated relative to the actual indication rate R1).

In addition, the characteristic measurement unit 318 of the information processing device 300 makes a determination on the speed selection (S190). Specifically, the characteristic measurement unit 318 compares the selected speed V2, which is the driving speed in the speed-variable display process P2 selected by the speed selection instruction VI, with the reference speed V1, which is the preset driving speed in the speed-fixed display process P1. The characteristic measurement unit 318 sets the value of the third coefficient k to “1” when the selected speed V2 is faster than the reference speed V1 (i.e., when the examinee EX makes a selection to increase the speed from the driving speed in the speed-fixed display process P1 in the speed selection after the speed-fixed display process P1) and sets the value of the third coefficient k to “−1” when the selected speed V2 is the reference speed V1 or less (i.e., when the examinee EX made a selection to maintain the driving speed in the speed-fixed display process P1 or to decrease the speed from the driving speed in the speed-fixed display process P1 in the speed selection after the speed-fixed display process P1).

The characteristic measurement unit 318 of the information processing device 300 makes a determination on the indication rate R2 in the speed-variable display process P2 (S200). Specifically, the characteristic measurement unit 318 calculates the indication rate R2 in the speed-variable display process P2 and compares the indication rate R2 with the reference indication rate Rs described above. The characteristic measurement unit 318 sets the value of the fourth coefficient l to “−1” when the indication rate R2 in the speed-variable display process P2 is below the reference indication rate Rs (i.e., when the indication rate R2 is relatively low) and sets the value of the fourth coefficient l to “0” when the indication rate R2 is the reference indication rate Rs or more (i.e., when the indication rate R2 is relatively high). In this embodiment, the value of the reference indication rate Rs used for determining the indication rate R2 in the speed-variable display process P2 is the same as the value of the reference indication rate Rs used for determining the indication rate R1 in the speed-fixed display process P1. However, the value of the reference indication rate Rs used for determining the indication rate R2 in the speed-variable display process P2 may be different from the value of the reference indication rate Rs used for determining the indication rate R1 in the speed-fixed display process P1. For example, considering that the simulated driving image SI is displayed for the second time in the speed-variable display process P2, the value of the reference indication rate Rs used for determining the indication rate R2 in the speed-variable display process P2 may be set to a value higher than the value of the reference indication rate Rs used for determining the indication rate R1 in the speed-fixed display process P1.

The determination processes from S170 to S200 do not necessarily need to be executed in the above order, and the execution order of these determination processes can be freely selected. Multiple determination processes may be executed in parallel.

Next, the characteristic measurement unit 318 of the information processing device 300 measures the characteristics of the examinee EX based on the combination of the first to fourth coefficients (i, j, k, l) set in the determination processes of S170 to S200 to generate characteristic information CHI indicating the characteristics of the examinee EX and output the characteristic information CHI (S210). As shown in FIGS. 6 and 7, in this embodiment, each combination of values of the first to fourth coefficients (i, j, k, l) is associated with different characteristics (24 different characteristics). The characteristic measurement unit 318 identifies the characteristics of the examinee EX based on the combination of values of the first to fourth coefficients (i, j, k, l) set in the determination process of S170 to S200, and displays the contents of the characteristic information CHI indicating the identified characteristics on the display unit 352.

In this embodiment, the characteristics of the examinee EX measured by the examinee-characteristic measurement process include hazard-perception ability, self-evaluation ability, self-control ability, and risk-taking tendency. The hazard-perception ability is the ability to detect hazards and is classified into, e.g., six levels (“very high”, “high”, “slightly high”, “slightly low”, “low”, and “very low”) based mainly on the results of the determination (S170, S200) on the indication rates R1, R2 in the speed-fixed display process P1 and/or the speed-variable display process P2 (i.e., the first coefficient i and/or the fourth coefficient l). For example, in the characteristic type “1” shown in FIG. 6, the indication rate R1 in the speed-fixed display process P1 is relatively high because the value of the first coefficient i is “0”, it is determined that the examinee EX made a selection to increase the speed from the speed (reference speed V1) in the speed-fixed display process P1 in selecting the speed (selected speed V2) for the speed-variable display process P2 because the value of the third coefficient k is “1”, and the indication rate R2 in the speed-variable display process P2 with increased speed is also relatively high because the value of the fourth coefficient l is “0”. Therefore, for the characteristic type “1” shown in FIG. 6, the hazard-perception ability is determined as “very high”. On the other hand, in the characteristic type “13” shown in FIG. 7, the indication rate R1 in the speed-fixed display process P1 is relatively high because the value of the first coefficient i is “0”, it is determined that the examinee EX made a selection to increase the speed from the speed (reference speed V1) in the speed-fixed display process P1 in selecting the speed (selected speed V2) for the speed-variable display process P2 because the value of the third coefficient k is “1”, and the indication rate R2 in the speed-variable display process P2 with increased speed is relatively low because the value of the fourth coefficient l is “−1”. Therefore, for the characteristic type “13” shown in FIG. 7, the hazard-perception ability is determined as “slightly high”. On the other hand, in the characteristic type “20” shown in FIG. 7, the indication rate R1 in the speed-fixed display process P1 is relatively low because the value of the first coefficient i is “−1”, it is determined that the examinee EX made a selection to decrease the speed (or maintain the speed) from the speed (reference speed V1) in the speed-fixed display process P1 in selecting the speed (selected speed V2) for the speed-variable display process P2 because the value of the third coefficient k is “−1”, and the indication rate R2 in the speed-variable display process P2 with increased speed is also relatively low because the value of the fourth coefficient l is “−1”. Therefore, for the characteristic type “20” shown in FIG. 7, the hazard-perception ability is determined as “quite low”.

The self-evaluation ability is the ability to objectively monitor one's own hazard-perception ability (i.e., meta-cognitive ability) and is classified into, e.g., three levels (“accurate”, “overestimated (overconfidence)”, and “underestimated (underconfidence)”) based mainly on the result of the determination (S180) on the self-evaluation SE of the indication rate (i.e., the second coefficient j). For example, in the characteristic type “1” shown in FIG. 6, the self-evaluation SE of the indication rate is close to the actual indication rate R1 because the value of the second coefficient j is “0”. Therefore, in the characteristic type “1” shown in FIG. 6, the self-evaluation ability is determined as “accurate”. On the other hand, in the characteristic type “3” shown in FIG. 6, the self-evaluation SE of the indication rate is excessive compared to the actual indication rate R1 because the value of the second coefficient j is “−1”. Therefore, in characteristic type “3” shown in FIG. 6, the self-evaluation ability is determined as “overestimated (overconfidence)”. In the characteristic type “5” shown in FIG. 6, the self-evaluation SE of the indication rate is underestimated compared to the actual indication rate R1 because the value of the second coefficient “j” is “1”. Therefore, in characteristic type “5” shown in FIG. 6, the self-evaluation ability is determined as “underestimated (underconfidence)”.

The self-control ability is the ability to select appropriate actions according to the movement environment based on the self-evaluation of the ability and is classified into, e.g., three levels (“high”, “normal”, and “low”) based mainly on the result of the determination (S170) on the indication rate R1 in the speed-fixed display process P1 (i.e., the first coefficient i), the result of the determination (S180) on the self-evaluation SE of the indication rate (i.e., the second coefficient j), and the result of the determination (S190) on the speed selection (i.e., the third coefficient k). For example, in the characteristic type “1” shown in FIG. 6, the indication rate R1 in the speed-fixed display process P1 is relatively high because the value of the first coefficient i is “0”, the self-evaluation SE of the indication rate is close to the actual indication rate R1 because the value of the second coefficient j is “0”, and it is determined that the examinee EX made a selection to increase the speed from the speed (reference speed V1) in the speed-fixed display process P1 in selecting the speed (selected speed V2) for the speed-variable display process P2 because the value of the third coefficient k is “1. In other words, the action to increase the speed is selected based on the high indication rate R1 and accurate self-evaluation. Therefore, in the characteristic type “1” shown in FIG. 6, the self-control ability is determined as “normal”. In the characteristic type “5” shown in FIG. 6, the indication rate R1 in the speed-fixed display process P1 is relatively high because the value of the first coefficient i is “0”, the self-evaluation SE of the indication rate is underestimated compared to the actual indication rate R1 because the value of the second coefficient “j” is “1”, and it is determined that the examinee EX made a selection to increase the speed from the speed (reference speed V1) in the speed-fixed display process P1 in selecting the speed (selected speed V2) for the speed-variable display process P2 because the value of the third coefficient k is “1”. In other words, the action to increase the speed is selected even though the self-evaluation SE is underestimated relative to the actual indication rate R1. Therefore, in the characteristic type “5” shown in FIG. 6, the self-control ability is determined as “low”.

The risk-taking tendency is the tendency to prefer taking risks and is classified, e.g., into two levels (“high” and “low”) based mainly on the result of the determination (S190) on the speed selection (i.e., the third coefficient k). For example, in the characteristic type “1” shown in FIG. 6, it is determined that the examinee EX made a selection to increase the speed from the speed (reference speed V1) in the speed-fixed display process P1 in selecting the speed (selected speed V2) for the speed-variable display process P2 because the value of the third coefficient k is “1. Therefore, in the characteristic type “1” shown in FIG. 6, the risk-taking tendency is determined as “high”. In the characteristic type “2” shown in FIG. 6, it is determined that the examinee EX made a selection to decrease the speed (or maintain the speed) from the speed (reference speed V1) in the speed-fixed display process P1 in selecting the speed (selected speed V2) for the speed-variable display process P2 because the value of the third coefficient k is “1”. Therefore, for the characteristic type “2” shown in FIG. 6, the risk-taking tendency is determined as “low”.

Various modes can be adopted as the output mode of the characteristic information CHI indicating the characteristics by the characteristic measurement unit 318 of the information processing device 300. For example, for the characteristic type assigned to the examinee EX, the determination result of each item of the characteristics illustrated in FIGS. 6 and 7 may be displayed on the display unit 352. Alternatively, a description of the characteristic determined based on the determination result of each item of the characteristic may be displayed on the display unit 352 (e.g., for characteristic type “1”, the description may be “You have a very high hazard-perception ability and an accurate self-evaluation ability. You have a high risk-taking tendency, but you have an average self-control ability). Alternatively, a code indicating the characteristic type assigned to the examinee EX (e.g., characteristic type “1”) may be displayed on the display unit 352, and the contents of the characteristic type may be communicated to examinee EX by other means (e.g., printed material distributed separately). The indication rate may be calculated for each type of hazard (pedestrian, bicycle, other vehicle, or the like), and if the pointing rate is low with a bias toward a particular type of hazard, an explanation may be displayed on the display unit 352 to communicate that the examinee has low cognitive ability for that type of hazard.

This completes the examinee-characteristic measurement process for measuring the characteristics of the examinee EX when the examinee EX drives a car and moves on a road.

### A-3. Effects of the First Embodiment

As explained above, the information processing device 300 of the first embodiment is a device for measuring the characteristics of the examinee EX when the examinee EX drives a car and moves along a road, and includes the speed selection instruction receiving unit 317, the display control unit 312, the location-pointing action accepting unit 316, and the characteristic measurement unit 318. The speed selection instruction receiving unit 317 receives a speed selection instruction VI issued by the examinee EX to select a driving speed. The display control unit 312 executes a speed-variable display process P2 to display on the display unit 352 a simulated moving image SI, which is a moving image simulating the field of vision of a human moving along a predetermined course and including at least one hazard Hn in such a manner that the driving speed is set in accordance with the speed selection instruction VI. The location-pointing action accepting unit 316 accepts the location-pointing action performed by the examinee EX to point to a specific location in the simulated driving image SI. The characteristic measurement unit 318 measures the characteristics of the examinee EX based on the indication rate R2 correlating with whether the location-pointing action correctly pointed to the location of the hazard Hn at a specific time when the scene including each hazard Hn in the simulated driving image SI is displayed in the speed-variable display process P2 and on the content of the speed selection instruction VI (which driving speed is selected) and outputs the characteristic information CHI representing the measurement results of the characteristics.

Thus, in the information processing device 300 of this embodiment, the display control unit 312 executes the speed-variable display process P2 in which the simulated driving image SI is displayed in such a manner that the driving speed is set according to the speed selection instructions VI received by the speed selection instruction receiving unit 317, and the characteristic measurement unit 318 measures the characteristics of the examinee EX based on the content of the speed selection instruction VI in addition to the indication rate R2 correlating with whether the location-pointing action correctly pointed to the location of the hazard Hn at the specific time when the scene including each hazard Hn of the simulated driving image SI is displayed in the speed-variable display process P2. Therefore, the information processing device 300 of this embodiment can perform the characteristic measurement for the examinee EX while considering the action of the examinee EX to select the driving speed of the car, thereby improving the appropriateness of the characteristic measurement for the examinee EX.

In the information processing device 300 of this embodiment, the display control unit 312 further executes a speed-fixed display process P1 to display the simulated driving image SI on the display unit 352 in a manner in which the driving speed is fixed to the preset reference speed V1. The characteristic measurement unit 318 measures the characteristics of the examinee EX based on the indication rate R1 in the speed-fixed display process P1, in addition to the indication rate R2 during the speed-variable display process P2 and the content of the speed selection instruction VI. Therefore, this information processing device 300 can measure the characteristics of the examinee EX by comparing the indication rate R1 and R2 in each of the speed-fixed display process P1, in which the driving speed is fixed to the reference speed V1, and the speed-variable display process P2, in which the driving speed can be selected by the examinee EX, thereby effectively improving the appropriateness of the characteristic measurement for the examinee EX.

Furthermore, in the information processing device 300 of this embodiment, the display control unit 312 executes the speed-variable display process P2 after the execution of the speed-fixed display process P1. The speed selection instruction receiving unit 317 receives the speed selection instruction VI after the execution of the speed-fixed display process P1 and before the execution of the speed-variable display process P2. Therefore, the information processing device 300 of this embodiment allows the examinee EX to select the driving speed (selected speed V2) in the speed-variable display process P2 based on the feeling (self-evaluation) of the degree of his/her own hazard recognition in the speed-fixed display process P1, and thereby accurately grasping the self-evaluation ability and the self-control ability of the examinee EX and effectively improving the appropriateness of the characteristic measurement for the examinee EX.

In the information processing device 300 of this embodiment, the characteristics of the examinee EX include the hazard-perception ability of the examinee EX and the self-control ability of the examinee EX. As described above, in the information processing device 300 of this embodiment, the characteristic measurement unit 318 measures the characteristics of the examinee EX based on the indication rate R2 during the speed-variable display process P2 and the content of the speed selection instruction VI. Therefore, the information processing device 300 of this embodiment can effectively improve the appropriateness of the characteristic measurement for the examinee EX, including the hazard-perception ability of the examinee EX and the self-control ability of the examinee EX.

In this embodiment of the information processing device 300, the characteristics of the examinee EX include the self-evaluation ability of the examinee EX. The information processing device 300 further includes the self-evaluation receiving unit 315 that receives a self-evaluation SE of the indication rate evaluated by the examinee EX. The characteristic measurement unit 318 measures the characteristics of the examinee EX based on the self-evaluation SE in addition to the indication rate R2 during the speed-variable display process P2 and the content of the speed selection instruction VI. Therefore, the information processing device 300 of this embodiment can measure the characteristics of the examinee EX based on the self-evaluation SE of the indication rate evaluated by the examinee EX, thereby effectively improving the appropriateness of the characteristics measurement for the examinee EX including the self-evaluation ability of the examinee EX. In the information processing device 300 of this embodiment, the characteristic measurement unit 318 determines the self-evaluation ability of the examinee EX based on the difference between the self-evaluation SE and the actual indication rate. Therefore, the information processing device 300 of this embodiment can further effectively improve the appropriateness of the characteristic measurement for the examinee EX, including the self-evaluation ability of the examinee EX.

The information processing device 300 of this embodiment includes a display unit 352 that displays a simulated driving image SI. Therefore, the information processing device 300 of this embodiment can simplify the configuration of the device for performing the characteristic measurement for the examinee EX.

### B. Second Embodiment

### B-1. Configuration of Information Processing Device 300 and Examinee-Characteristic Measurement Process

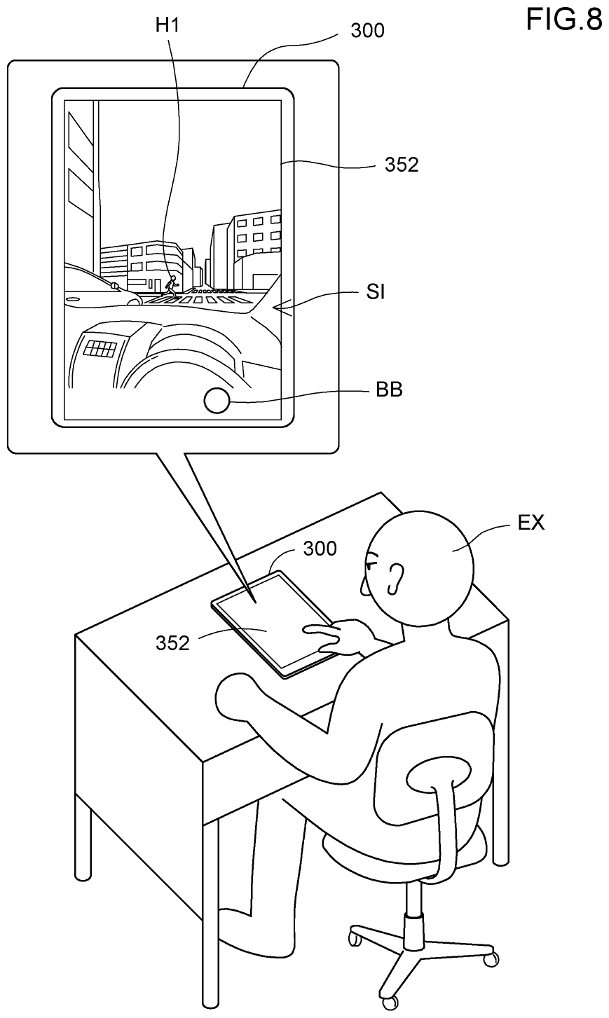

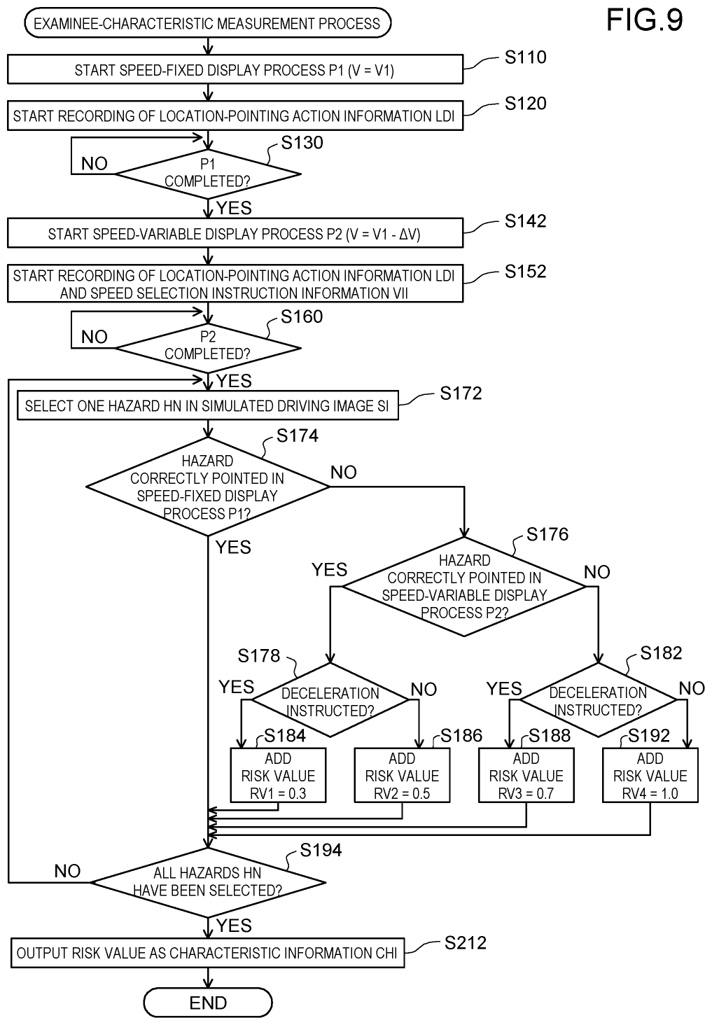

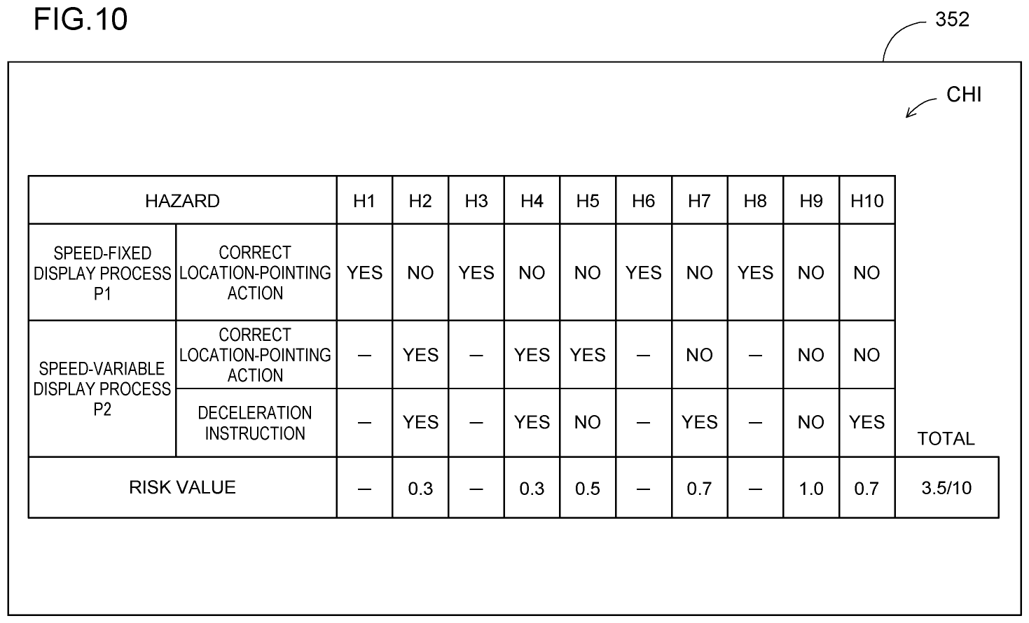

FIG. 8 is an explanatory diagram illustrating a schematic configuration of an information processing device 300 in the second embodiment, FIG. 9 is a flowchart illustrating the contents of an examinee-characteristic measurement process executed by the information processing device 300 in the second embodiment, and FIG. 10 is an explanatory diagram illustrating an example of a state in which characteristic information CHI indicating the result of the examinee-characteristic measurement process in the second embodiment is displayed on a display unit 352. In the following, among the components of the information processing device 300 and the contents of the examinee-characteristic measurement process in the second embodiment, those identical to those of the first embodiment described above will be omitted by labeling the same symbols as appropriate.

In the examinee-characteristic measurement process in the first embodiment described above, the speed selection instruction receiving unit 317 of the information processing device 300 receives the speed selection instruction VI after execution of the speed-fixed display process P1 and before execution of the speed-variable display process P2. On the other hand, in the examinee-characteristic measurement process in the second embodiment, the speed selection instruction receiving unit 317 receives the speed selection instruction VI during the speed-variable display process P2. In other words, the examinee is instructed to touch the deceleration button BB (FIG. 8) displayed on the display unit 352 as the speed selection instruction VI to decrease the driving speed (hereinafter also referred to as the “deceleration instruction”) when the examinee EX wishes to decrease the driving speed of the car during the speed-variable display process P2.

In the examinee-characteristic measurement process in the second embodiment, among the characteristics of the examinee EX, the risk when the examinee EX drives a car and moves on a road is measured.

In the examinee-characteristic measurement process in the second embodiment shown in FIG. 9, the processes from S110 to S130 (processes related to the speed-fixed display process P1) of the examinee-characteristic measurement process in the first embodiment shown in FIG. 3 are executed in the same manner.

After completion of the speed-fixed display process P1, the display control unit 312 of the information processing device 300 starts the speed-variable display process P2 (S142). In the speed-variable display process P2, the display control unit 312 causes the simulated driving image SI to be displayed in such a manner that the driving speed V is set according to the speed selection instruction VI issued by the examinee EX. Specifically, the speed selection instruction receiving unit 317 monitors whether a deceleration instruction (touch operation to the deceleration button BB displayed on the display unit 352) is received from the examinee EX during the speed-variable display process P2, and if such a deceleration instruction is received, the display control unit 312 switches the simulated driving image SI displayed on the display unit 352 to a simulated driving image SI that simulates the view from the driver's seat of a car whose driving speed has decreased from the reference speed V1. The driving speed of the car in the simulated driving image SI may be changed in two steps (reference speed V1 and low speed (V1−ΔV)), or in three or more steps or steplessly (continuously).

When the speed-variable display process P2 is started, as in the speed-fixed display process P1 described above, the location-pointing action accepting unit 316 of the information processing device 300 starts accepting location-pointing actions performed by the examinee EX to point to a specific location in the simulated driving image SI and starts recording the location-pointing action information LDI indicating the history of such location-pointing actions (S152). At the same time, the speed selection instruction receiving unit 317 of the information processing device 300 starts accepting deceleration instructions from the examinee EX and starts recording the speed selection instruction information VII, which indicates the history of the deceleration instructions. The speed selection instruction information VII is information indicating the time when the examinee EX issues the deceleration instruction (time point in the simulated driving image SI). By referring to the speed selection instruction information VII, it is possible to know at which scene in the simulated driving image SI the examinee EX felt enough risk to need to decelerate. The received and updated location-pointing action information LDI and speed selection instruction information VII are stored in the storage unit 330.

The display control unit 312 of the information processing device 300 monitors whether the speed-variable display process P2 is completed (S160), and if it is determined that the speed-variable display process P2 is completed (S160: YES), the process proceeds to S172.

When the speed-fixed display process P1 and the speed-variable display process P2 are completed, the characteristic measurement unit 318 of the information processing device 300 refers to the correct answer information RAI stored in advance in the storage unit 330, and the location-pointing action information LDI and the speed selection instruction information VII generated and updated during execution of the speed-fixed display process P1 and the speed-variable display process P2, and starts the risk measurement as one of the characteristic measurements for the examinee EX while driving, as detailed below.

First, the characteristic measurement unit 318 of the information processing device 300 selects one hazard Hn in the simulated driving image SI (S172), and with reference to the correct answer information RAI and the location-pointing action information LDI, determines whether the examinee EX correctly pointed to the location of the hazard Hn (touched the location where the hazard Hn is displayed on the display unit 352) at a specific time when the scene including the selected hazard Hn is displayed during the speed-fixed display process P1 (S174). This determination is made by referring to the correct answer information RAI to identify the display timing of the scene containing the selected hazard Hn (display time of the frame containing the hazard Hn) and the location of the hazard Hn (coordinates of the image area representing the hazard Hn on the frame) and by referring to the location-pointing action information LDI, to determine whether a location-pointing action is made to specify the location of the hazard Hn at the display timing. If it is determined in S174 that there was a location-pointing action that correctly points the location of the hazard Hn during the speed-fixed display process P1 (S174: YES), the characteristic measurement unit 318 determines that the examinee EX could correctly recognize the hazard Hn (this case is hereinafter referred to as “hazard-recognized case C0”), and the process proceeds to S194 without executing the process of adding risk values as described below. In the example of the characteristic measurement results shown in FIG. 10, for each of hazards H1, H3, H6, and H8, since examinee EX could correctly recognize the hazard during the speed-fixed display process P1 (correct location-pointing action: YES), the risk value is not added (in FIG. 10, the symbol “-” is shown in the risk value column).

On the other hand, if it is determined in S174 that there was no location-pointing action that correctly specifies the location of hazard Hn during the speed-fixed display process P1 (S174: NO), the characteristic measurement unit 318 of the information processing device 300 refers to the correct answer information RAI and the location-pointing action information LDI to determine whether there was a location-pointing action performed by the examinee EX correctly pointing to the location of hazard Hn at the specific time when the scene including the selected hazard Hn is displayed during the speed-variable display process P2 (S176). If it is determined in S176 that there was no location-pointing action to correctly point to the location of the hazard Hn during the speed-variable display process P2 (S176: NO), that is, if the examinee EX failed to correctly recognize the hazard Hn during the speed-fixed display process P1 and the speed-variable display process P2, the characteristic measurement unit 318 refers to the speed selection instruction information VII to determine whether a deceleration instruction (touch operation to the deceleration button BB displayed on the display unit 352) is issued by the examinee EX at the time when the scene including the hazard Hn is displayed during the speed-variable display process P2 (S182).

If it is determined in S182 that there was a deceleration instruction from the examinee EX at the specific time when the scene including the hazard Hn was displayed during the speed-variable display process P2 (S182: YES), the characteristic measurement unit 318 of the information processing device 300 determines that although the examinee EX failed to correctly recognize the hazard Hn during the speed-fixed display process P1 and the speed-variable display process P2, the examinee EX issued a deceleration instruction based on an objective understanding (metacognition) of his/her own recognition ability (this case is hereinafter referred to as “no-hazard-recognized/with deceleration case C3”), and adds the third risk value Rv3 (e.g., Rv3=0.7) (S188). In the example of the characteristic measurement results shown in FIG. 10, for each of hazards H7 and H10, although the examinee EX failed to correctly recognize the hazard during the speed-fixed display process P1 and the speed-variable display process P2 (correct location-pointing action: NO), the examinee EX issued a deceleration instruction in the scene containing the hazard during the speed-variable display process P2 (deceleration instruction: YES) so that the third risk value Rv3 (e.g., Rv3=0.7) associated with the no-hazard-recognized/with deceleration case C3 is added.

On the other hand, if it is determined in S182 that there was no deceleration instruction from the examinee EX at the specific time when the scene including the hazard Hn is displayed during the speed-variable display process P2 (S182: NO), the characteristic measurement unit 318 of the information processing device 300 determines that the examinee EX did not correctly recognize the hazard Hn during the speed-fixed display process P1 and the speed-variable display process P2, and did not issue a deceleration instruction because he/she did not objectively grasp (metacognition) his/her own recognition ability (this case is hereinafter referred to as “no-hazard-recognized/without deceleration case C4”), and adds the fourth risk value Rv4 (e.g., Rv4=1.0) (S192). The fourth risk value Rv4 associated with this no-hazard-recognized/without deceleration case C4 is set to a value greater than the third risk value Rv3 associated with the no-hazard-recognized/with deceleration case C3 described above. In the example of the characteristic measurement result shown in FIG. 10, for hazard H9, since examinee EX failed to correctly recognize the hazard during the speed-fixed display process P1 and the speed-variable display process P2 (correct location-pointing action: NO) and did not issue a deceleration instruction in the scene containing the hazard during the speed-variable display process P2 (deceleration instruction: NO), the fourth risk value Rv4 (e.g., Rv4=1.0) associated with the no-hazard-recognized/without deceleration case C4, is added.

When it is determined in S176 that the examinee EX correctly pointed to the location of the hazard Hn during the speed-variable display process P2 (S176: YES), that is, although the examinee EX failed to correctly recognize the hazard Hn during the speed-fixed display process P1, the examinee EX could correctly recognize the hazard Hn during the speed-variable display process P2, the characteristic measurement unit 318 refers to the speed selection instruction information VII to determine whether there was a deceleration instruction from the examinee EX at the specific time when the scene including the hazard Hn is displayed during the speed-variable display process P2 (S178).

If it is determined in S178 that there was a deceleration instruction from the examinee EX at the specific time when the scene including the hazard Hn was displayed during the speed-variable display process P2 (S178: YES), the characteristic measurement unit 318 of the information processing device 300 determines that the examinee EX could correctly recognize the hazard Hn during the speed-variable display process P2, which could not be correctly recognized during the speed-fixed display process P1 and issued the deceleration instruction based on an objective understanding (metacognition) of his/her own recognition ability (this case is hereinafter referred to as “hazard-partly-recognized/with deceleration case C1”), and adds the first risk value Rv1 (e.g., Rv1=0.3) (S184). In the example of the characteristic measurement results shown in FIG. 10, for each of hazards H2 and H4, although the examinee EX failed to correctly recognize the hazard during the speed-fixed display process P1 (correct location-pointing action: NO), the examinee EX could correctly recognize the hazard during the speed-variable display process P2 (correct location-pointing action: YES) and issued the deceleration instruction in the scene including the hazard during the speed-variable display process P2 (deceleration instruction: YES) so that the first risk value Rv1 (e.g., Rv1=0.3) associated with the hazard-partly-recognized/with deceleration case C1 is added.

On the other hand, if it is determined in S178 that there was no deceleration instruction from the examinee EX at the specific time when the scene including the hazard Hn is displayed during the speed-variable display process P2 (S178: NO), the characteristic measurement unit 318 of the information processing device 300 determines that although the examinee EX could correctly recognize the hazard Hn during the speed-variable display process P2, which could not be correctly recognized during the speed-fixed display process P1, the examinee EX did not issue a deceleration instruction due to the lack of the objective understanding (metacognition) his/her own recognition ability (this case is hereinafter referred to as “hazard-partly-recognized/without deceleration case C2”), and adds the second risk value Rv2 (e.g., Rv2=0.5) (S186). This second risk value Rv2 associated with the hazard-partly-recognized/without deceleration case C2 is smaller than the third risk value Rv3 associated with the no-hazard-recognized/with deceleration case C3 described above but larger than the first risk value, Rv1, associated with hazard-partly-recognized/with deceleration case C1. In the example of the characteristic measurement results shown in FIG. 10, for hazard H5, although the examinee EX failed to correctly recognize the hazard during the speed-fixed display process P1 (correct location-pointing action: NO), the examinee EX could correctly recognize the hazard during the speed-variable display process P2 (correct location-pointing action: YES) but did not issue the deceleration instruction in the scene containing the hazard during the speed-variable display process P2 (deceleration instruction: NO), the second risk value Rv2 (e.g., Rv2=0.5) associated with the hazard-partly-recognized/without deceleration case C2 is added.

When the processes of S174 to S192 described above are completed for one selected hazard Hn, the characteristic measurement unit 318 determines whether all hazards Hn in the simulated driving image SI have been selected (S194), and if it is determined that one or more unselected hazards Hn remain (S194: NO), the process returns to the hazard Hn selection process (S172), and subsequent processes are performed in the same manner. In the example of evaluation results shown in FIG. 10, since there are ten hazards Hn, the above-mentioned processes S174 to S192 are repeatedly executed for each hazard Hn until it is determined that the selection of ten hazards Hn is completed.

After repeating these processes, when it is determined that the selection of all hazards Hn is completed at S194 (S194: YES), the characteristic measurement unit 318 generates characteristic information CHI that represents the result of the characteristic measurement (e.g., the total risk value score) and outputs the characteristic information CHI (S212). For example, the characteristic measurement unit 318 displays the contents of the characteristic information CHI on the display unit 352, as shown in FIG. 10. The above completes the examinee-characteristic measurement process for evaluating the risk when the examinee EX drives a car and moves on a road.

In the example of the characteristic measurement result shown in FIG. 10, no risk value is added for four hazards (hazards H1, H3, H6, and H8) as being in the hazard-recognized case C0, the first risk value Rv1 (e.g., Rv1=0.3) is added for two hazards (hazards H2 and H4) as being in the hazard-partly-recognized/with deceleration case C1, the second risk value Rv2 (e.g., Rv2=0.5) is added for one hazard (hazard H5) as being in the hazard-partly-recognized/without deceleration case C2, the third risk value Rv3 (e.g., Rv3=0.7) is added for two hazards (hazards H7 and H10) as being in the no-hazard-recognized/with deceleration case C3, the fourth risk value Rv4 (e.g., Rv4=1.0) is added for one hazard (hazard H9) as being in no-hazard-recognized/without deceleration case C4 so that the total risk value is for example 3.5 points (the maximum risk value is for example 10 points). The higher the risk value, the higher the risk for the examinee EX to drive a car and move on the road.

### B-2. Effects of the Second Embodiment

As explained above, in the information processing device 300 of the second embodiment, as in the information processing device 300 of the first embodiment, the display control unit 312 executes the speed-variable display process P2 in which the simulated driving image SI is displayed in such a manner that the driving speed is set according to the speed selection instructions VI received by the speed selection instruction receiving unit 317, and the characteristic measurement unit 318 measures the characteristics of the examinee EX based on the content of the speed selection instruction VI (presence or absence of the speed selection instruction VI to reduce driving speed) in addition to the indication results correlating with whether the location-pointing action correctly pointed to the location of the hazard Hn at a specific time when the scene including each hazard Hn in the simulated moving image SI is displayed in the speed-variable display process P2. Therefore, the information processing device 300 of the second embodiment can perform the characteristic measurement for the examinee EX while considering the action of the examinee EX to select the driving speed of the car, thereby improving the appropriateness of the characteristic measurement for the examinee EX.

In the information processing device 300 of the second embodiment, as in the information processing device 300 of the first embodiment, the display control unit 312 further executes the speed-fixed display process P1 to display the simulated driving image SI on the display unit 352 in a manner in which the driving speed is fixed to the preset reference speed V1, and the characteristic measurement unit 318 measures the characteristics of the examinee EX based on the indication results in the speed-fixed display process P1 in addition to the indication results in the speed-variable display process P2 and the content of the speed selection instruction VI. Therefore, the information processing device 300 of the second embodiment can measure the characteristics of the examinee EX by comparing the indication results in each of the speed-fixed display process P1, in which the driving speed is fixed to the reference speed V1, and the speed-variable display process P2, in which the driving speed can be selected by the examinee EX, thereby effectively improving the appropriateness of the characteristic measurement for the examinee EX.

Furthermore, in the information processing device 300 of the second embodiment, the speed selection instruction receiving unit 317 receives the speed selection instruction VI during the speed-variable display process P2, and the display control unit 312 changes the driving speed according to the speed selection instruction VI during the speed-variable display process P2. Therefore, the information processing device 300 of the second embodiment can perform the characteristic measurement for the examinee EX while considering the action closer to the actual driving environment, such as changing the driving speed (decelerating) while driving a car, thereby effectively improving the appropriateness of the characteristic measurement for the examinee EX.

Furthermore, in the information processing device 300 of the second embodiment, the characteristics of the examinee EX include the risk of the examinee EX while moving, and in a case where there is no location-pointing action correctly pointing to the location of hazard Hn at the above specific time during the speed-variable display process P2, the characteristic measurement unit 318 determines that the risk is lower when there is a deceleration instruction than when there is no deceleration instruction. In a case where the examinee EX failed to recognize the hazard Hn at the above specific time during the speed-variable display process P2, when there is a deceleration instruction, it is assumed that the examinee EX took compensatory action due to metacognition of his/her own cognitive deficiency, while when there is no deceleration instruction, it is assumed that the examinee EX did not take compensatory action due to a lack of metacognition of his/her own cognitive deficiency. The information processing device 300 of the second embodiment can determine the risk when such a compensatory action is taken to be lower than when no compensatory action is taken, thereby improving the appropriateness of the risk measurement as the characteristic measurement for the examinee EX.

Also, in the information processing device 300 of the second embodiment, the characteristic measurement unit 318 determines that in a case where there is no location-pointing action correctly pointing to the location of the hazard Hn at the above specific time during the speed-fixed display process P1, and there is no location-pointing action correctly pointing to the location of the hazard Hn at the above specific time during the speed-variable display process P2, the risk is lower when there is a deceleration instruction than when there is no deceleration instruction during the speed-variable display process P2. When the driver failed to recognize the hazard Hn during either the speed-fixed display process P1 and the speed-variable display process P2, when there is a deceleration instruction, it is assumed that the examinee took compensatory action due to metacognition of his/her own cognitive deficiency, while when there is no deceleration instruction, it is assumed that the examinee did not take compensatory action due to a lack of metacognition of his/her own cognitive deficiency. The information processing device 300 of the second embodiment can determine the risk when such a compensatory action is taken to be lower than when no compensatory action is taken, thereby improving the appropriateness of the risk measurement as the characteristic measurement for the examinee EX.

In the information processing device 300 of the second embodiment, the characteristic measurement unit 318 determines that in a case where there is no location-pointing action correctly pointing to the location of the hazard Hn at the above specific time during the speed-fixed display process P1, and there is a location-pointing action correctly pointing to the location of the hazard Hn at the above specific time during the speed-variable display process P2, the risk is lower when there is a deceleration instruction than when there is no deceleration instruction during the speed-variable display process P2. Even in a case where the examinee could recognize the hazard Hn during the speed-variable display process P2 but failed to recognize the hazard Hn during the speed-fixed display process P1, when there is a deceleration instruction, it is assumed that the examinee took compensatory action due to metacognition of his/her own cognitive deficiency, while when there is no deceleration instruction, it is assumed that the examinee did not take compensatory action due to a lack of metacognition of his/her own cognitive deficiency. The information processing device 300 of the second embodiment can determine the risk when such a compensatory action is taken to be lower than when no compensatory action is taken, thereby improving the appropriateness of the risk measurement as the characteristic measurement for the examinee EX.

Furthermore, in the information processing device 300 of this embodiment, the display control unit 312 executes the speed-variable display process P2 after the execution of the speed-fixed display process P1. Therefore, the information processing device 300 of this embodiment can accurately grasp the level of the metacognitive ability of the examinee based on the presence or absence of the deceleration instruction in the subsequent speed-variable display process P2 for a scene containing a hazard Hn that could not be recognized in the speed-fixed display process P1, thereby effectively improving the appropriateness of the characteristic measurement for the examinee EX.

### C. Third Embodiment

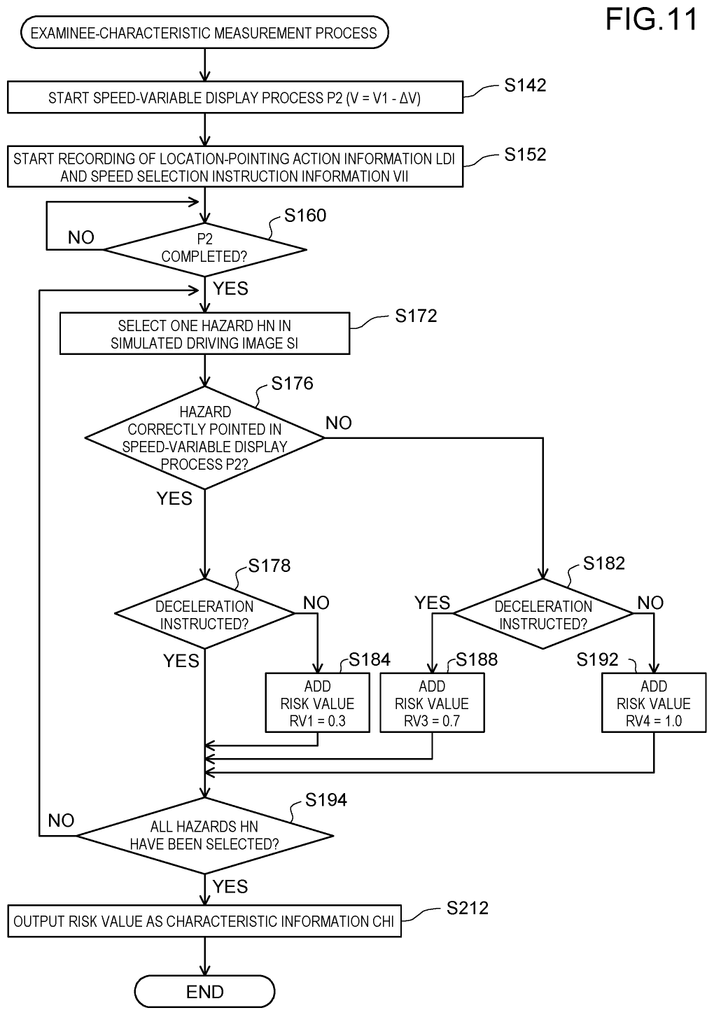

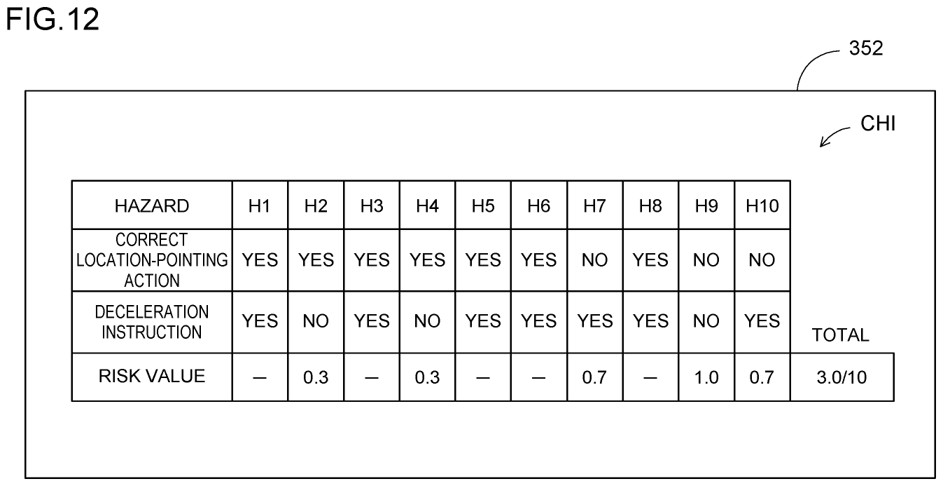

FIG. 11 is a flowchart illustrating the contents of an examinee-characteristic measurement process in the third embodiment. FIG. 12 is an explanatory diagram illustrating an example of a state in which characteristic information CHI indicating the result of the examinee-characteristic measurement process in the third embodiment is displayed on the display unit 352. The configuration of the information processing device 300 of the third embodiment is identical to the configuration of the information processing device 300 of the second embodiment described above. In the following, among the processing contents of the examinee-characteristic measurement process in the third embodiment, the same processing contents as in the second embodiment described above will be omitted by labeling the same symbols as appropriate.

In the examinee-characteristic measurement process in the second embodiment described above, two processes, the speed-fixed display process P1 and the speed-variable display process P2, are executed as the display (reproduction) process of the simulated driving image SI, but in the examinee-characteristic measurement process in the third embodiment, only the speed-variable display process P2 is executed without execution of the speed-fixed display process P1. In other words, in the examinee-characteristic measurement process in the third embodiment shown in FIG. 11, the processes from S110 to S130 (processes related to the speed-fixed display process P1) of the examinee-characteristic measurement process in the second embodiment shown in FIG. 9 are not executed, and the process starts from S142. In the examinee-characteristic measurement process in the third embodiment, in the characteristic (risk) measurement process after S172, the process of S176 is executed immediately without executing the process of S174 shown in FIG. 9 (process for determining whether the hazard is recognized in the speed-fixed display process P1).

In the examinee-characteristic measurement process in the third embodiment, when it is determined that there was no location-pointing action that correctly points the location of hazard Hn during the speed-variable display process P2 (S176: NO), the process is the same as the examinee-characteristic measurement process in the second embodiment. That is, when it is determined in S176 that there was no location-pointing action that correctly points the location of hazard Hn during the speed-variable display process P2 (S176: NO), the characteristic measurement unit 318 determines whether a deceleration instruction was issued by the examinee EX at the time when the scene including the hazard Hn is displayed (S182), and when there was a deceleration instruction (S182: YES), the third risk value Rv3 (e.g., Rv3=0.7) is added (S188), and when there was no deceleration instruction (S182: NO), the fourth risk value Rv4 (e.g., Rv4=1.0) is added (S192). In the example of the characteristic measurement results shown in FIG. 12, for each of hazards H7 and H10, although the examinee EX failed to correctly recognize the hazard during the speed-variable display process P2 (correct location-pointing action: NO), the examinee EX issued a deceleration instruction in the scene containing the hazard (deceleration instruction: YES) so that the third risk value Rv3 (e.g., Rv3=0.7) is added, and for Hazard H9, the examinee EX failed to correctly recognize the hazard during the speed-variable display process P2 (correct location-pointing action: NO) and did not issue a deceleration instruction in the scene containing the hazard (deceleration instruction: NO) so that the fourth risk value Rv4 (e.g., Rv4=1.0) is added.

On the other hand, in the examinee-characteristic measurement process in the third embodiment, when it is determined that there was a location-pointing action that correctly points the location of the hazard Hn during the speed-variable display process P2 (S176: YES), the process differs from the examinee-characteristic measurement process in the second embodiment. That is, in this case, the characteristic measurement unit 318 determines whether there is a deceleration instruction from the examinee EX at the time when the scene including the hazard Hn is displayed (S178). If it is determined that there was a deceleration instruction (S178: YES), the characteristic measurement unit 318 determines that the examinee EX has decelerated the car, does not execute the process of adding risk values, and proceeds to S194. In the example of the characteristic measurement results shown in FIG. 12, for each of hazards H1, H3, H5, H6, and H8, since the examinee EX issued a deceleration instruction during the speed-variable display process P2 (deceleration instruction: YES), which means that the examinee EX correctly recognized the hazard (correct location-pointing action: YES), the risk value is not added.

On the other hand, if it is determined that there was no deceleration instruction (S178: NO), the characteristic measurement unit 318 determines that the examinee EX did not issue a deceleration instruction although the hazard was correctly recognized so that the first risk value Rv1 (e.g., Rv1=0.3), which is smaller than the third and fourth risk values Rv3 and Rv4 described above, is added (S184). In the example of the characteristic measurement results shown in FIG. 12, for each of hazards H2 and H4, since the examinee EX could correctly recognize the hazard during the speed-variable display process P2 (correct location-pointing action: YES) but did not issue a deceleration instruction (deceleration instruction: NO), the first risk value Rv1 (e.g., Rv1=0.3) is added. Subsequent processes are the same as in the second embodiment.

As explained above, also in the information processing device 300 of the third embodiment, the display control unit 312 executes the speed-variable display process P2 in which the simulated driving image SI is displayed in such a manner that the driving speed is set according to the speed selection instruction VI received by the speed selection instruction receiving unit 317, and the characteristic measurement unit 318 measures the characteristics of the examinee EX based on the content of the speed selection instruction VI in addition to the indication results correlating with whether the location of the hazard Hn is correctly pointed to at the specific time when the scene including the hazard Hn of the simulated driving image SI is displayed during the speed-variable display process P2. Therefore, the information processing device 300 of the third embodiment, as with the information processing device 300 of the second embodiment, can perform the characteristic measurement for the examinee EX while considering the action of the examinee EX to select the driving speed of the car, thereby improving the appropriateness of the characteristic measurement for the examinee EX.

### D. Fourth Embodiment

### D-1. Configuration of Information Processing System 10

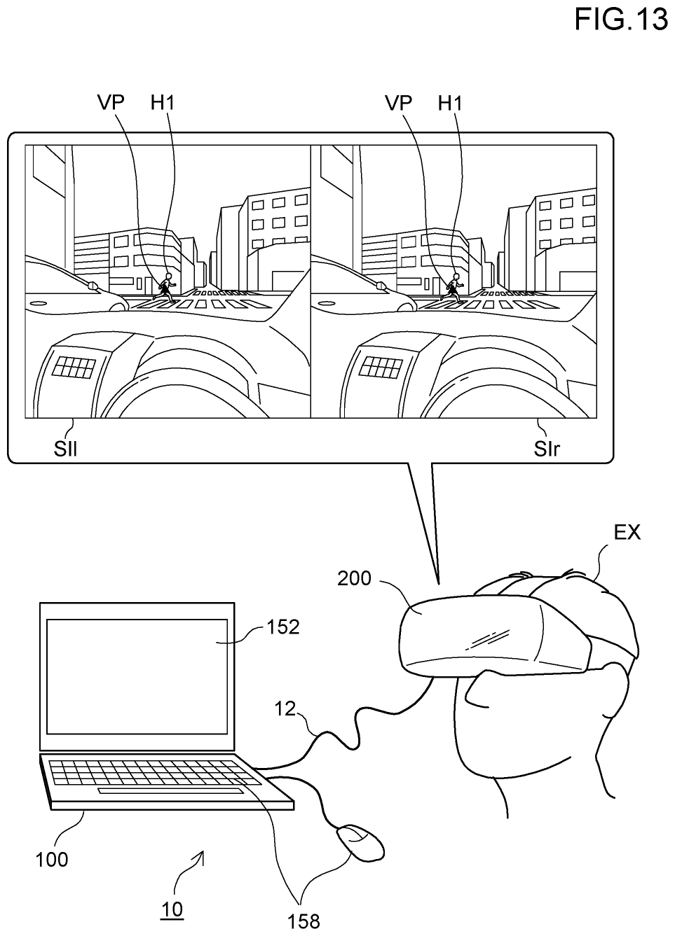

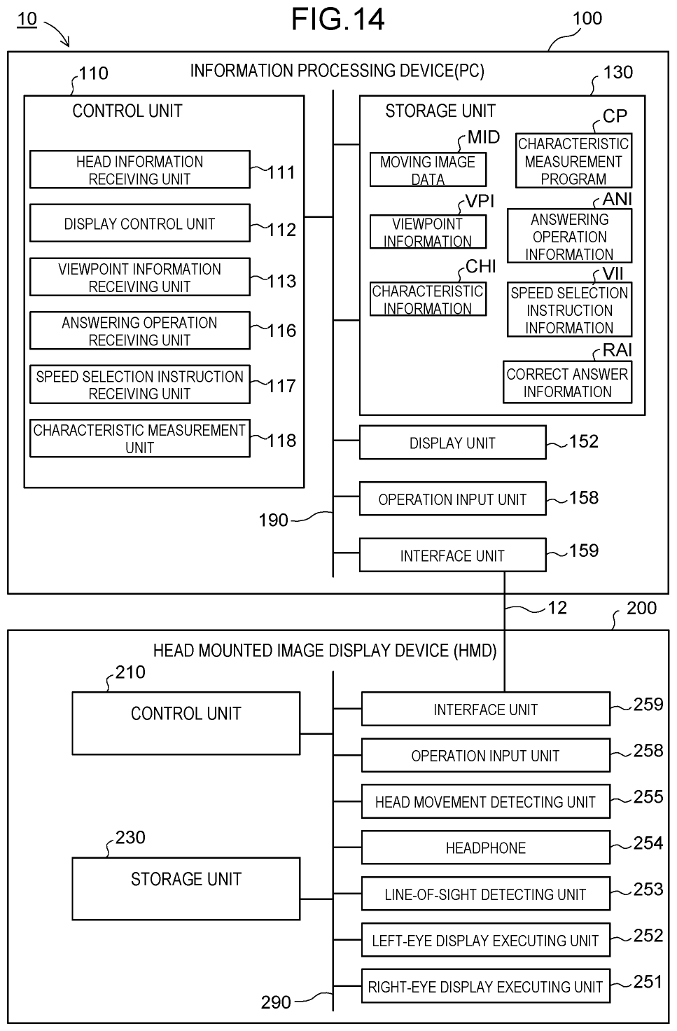

FIG. 13 is an explanatory diagram illustrating a schematic configuration of an information processing system 10 in the fourth embodiment, and FIG. 14 is a block diagram illustrating a schematic configuration of an information processing system 10 in the fourth embodiment. In the following, among the components of the fourth embodiment, the same components as those of the second embodiment described above will be omitted from the description as appropriate by labeling the same symbols.

The information processing system 10 of the fourth embodiment is a system for measuring the characteristics of an examinee EX when the examinee EX drives a car and moves along a road. More specifically, the information processing system 10 is a system that causes the examinee EX to view a simulated driving image SI (image SIr for the right eye and image SIl for the left eye) that simulates the field of vision of a human driving a car and moving along a predetermined course and performs a hazard recognition test to determine whether the examinee EX has recognized each hazard included in the simulated driving image SI to measure the characteristics of the examinee EX when he/she drives a car and moves on a road based on the results of the hazard recognition test. As shown in FIGS. 13 and 14, the information processing system 10 includes an information processing device 100, and a head mounted image display device (hereinafter referred to as “HMD”) 200 as an image display device.

### Configuration of Information Processing Device 100

As shown in FIGS. 13 and 14, a personal computer (hereinafter referred to as a “PC”) is used as the information processing device 100 in this embodiment. The information processing device 100 includes a control unit 110, a storage unit 130, a display unit 152, an operation input unit 158, and an interface unit 159. These units are communicably connected to each other via a bus 190.

The display unit 152 of the information processing device 100 is composed of, e.g., a liquid crystal display, an organic EL display, or the like, and displays various images and information. The operation input unit 158 of the information processing device 100 is composed of, e.g., a keyboard, a mouse, a microphone, or the like, and receives operations and instructions from the administrator or examinee EX. The interface unit 159 of the information processing device 100 is configured by, e.g., a LAN interface, a USB interface, or the like, and communicates with other devices in a wired or wireless manner. In this embodiment, the interface unit 159 of the information processing device 100 is connected to the interface unit 259 of the HMD 200 (see below) via the cable 12, and communicates with the interface unit 259 of the HMD 200.

The storage unit 130 of the information processing device 100 is composed of, e.g., ROM, RAM, hard disk drive (HDD), or the like and is used for storing various programs and data, and as a work area for executing various programs, and as a temporary storage area for data. For example, the storage unit 130 stores the characteristic measurement program CP, which is a computer program for executing the risk evaluation process during operation as described below.

Moving image data MID is stored in the storage unit 130 of the information processing device 100. In this embodiment, the moving image data MID includes data of a plurality of simulated driving images SI associated with each orientation of the head of the examinee EX to change the simulated driving images SI to be viewed by the examinee EX according to the movement of the head of the examinee EX, as will be described later. In this embodiment, the simulated driving image SI consists of an image SIr for the right eye and an image SIl for the left eye, which are generated in consideration of parallax so that the simulated driving image SI to be viewed by the examinee EX is a 3D image. The moving image data MID representing the simulated driving image SI may, e.g., be generated by 3D-CG software or by using images captured by an omnidirectional camera while driving a car on a real road.

The storage unit 130 of the information processing device 100 stores viewpoint information VPI, answering operation information ANI, speed selection instruction information VII, and characteristic information CHI during the examinee-characteristic measurement process described below. The contents of these pieces of information are explained in conjunction with the description of the examinee-characteristic measurement process described below.

The control unit 110 of the information processing device 100 is composed of, e.g., a CPU or the like, and controls the operation of the information processing device 100 by executing a computer program loaded from the storage unit 130. For example, the control unit 110 executes the examinee-characteristic measurement process described below by loading the characteristic measurement program CP from the storage unit 130 and executing it. In more detail, the control unit 110 functions as a head information receiving unit 111, a display control unit 112, a viewpoint information receiving unit 113, an answering operation receiving unit 116, a speed selection instruction receiving unit 117, and a characteristic measurement unit 118 to execute the examinee-characteristic measurement process described below. The functions of each of these units are explained in conjunction with the description of the examinee-characteristic measurement process described below.

### Configuration of HMD 200

The HMD 200 served as an image display device is a device for causing the examinee EX to view images while being worn on the head of the examinee EX. The HMD 200 of this embodiment is a non-transmissive head-mounted display that completely covers both eyes of the examinee EX and can provide a virtual reality (VR) function. In this specification, causing the examinee EX to view an image by the HMD 200 is also expressed as displaying an image (to the examinee EX) by the HMD 200. The HMD 200 is an example of an image display unit in the claims.

The HMD 200 includes a control unit 210, a storage unit 230, a right-eye display executing unit 251, a left-eye display executing unit 252, a line-of-sight detecting unit 253, a headphone 254, a head movement detecting unit 255, an operation input unit 258, and an interface unit 259. These units are communicatively connected to each other via a bus 290.

The right-eye display executing unit 251 of the HMD 200 includes, for example, a light source, a display element (digital mirror devices (DMD), liquid crystal panels, and the like), and an optical system, and generates image light representing a right eye image SIr constituting the simulated driving image SI and guides the image light to the right eye of the examinee EX, thereby causing the right eye of the examinee EX to view the right eye image SIr. The left-eye display executing unit 252 is provided independently of the right-eye display executing unit 251, and similarly to the right-eye display executing unit 251, includes, for example, a light source, a display element, and an optical system, generates image light representing a left eye image SIL constituting the simulated driving image SI and guides the generated image light to the left eye of the examinee EX, thereby causing the left eye of the examinee EX to view the left eye image SIL. In the state in which the right eye of the examinee EX views the right eye image SIr and the left eye of the examinee EX views the left eye image SIl, the examinee EX views the simulated driving image SI as a 3D image.

The line-of-sight detecting unit 253 of the HMD 200 detects the line-of-sight of the examinee EX in order to implement a so-called eye tracking function. For example, the line-of-sight detecting unit 253 includes a light source for emitting non-visible light and a camera, emits non-visible light from the light source, images the non-visible light reflected by the eye of the examinee EX by the camera to generate an image, and analyzes the generated image to detect the line-of-sight direction of the examinee EX. The line-of-sight detecting unit 253 repeatedly executes detection of the line-of-sight direction at a predetermined frequency (for example, at a frequency corresponding to the frame rate of the moving image displayed by the right-eye display executing unit 251 and the left-eye display executing unit 252). It should be noted that the line-of-sight detecting unit 253 can specify the location of the viewpoint VP (see FIG. 13) of the examinee EX on the image that the examinee EX is viewing by detecting the line-of-sight of the examinee EX.

The headphone 254 of the HMD 200 is a device that is placed on the ears of the examinee EX and outputs sound. The head movement detecting unit 255 of the HMD 200 is a sensor for detecting movement of the HMD 200 (that is, the movement of the head of the examinee EX) to implement a so-called head tracking function. The movement of the head of the examinee EX is a concept including a change in the location and direction of the head of the examinee EX. The operation input unit 258 of the HMD 200 includes, for example, a button for receiving instructions from the examinee EX. The operation input unit 258 may be disposed inside the housing (the part mounted on the head of the examinee EX) of the HMD 200 or may be configured as a separate component connected to the housing via a signal line. The interface unit 259 of the HMD 200 includes, for example, a LAN interface or a USB interface and communicates with other devices through wired or wireless connection.

The storage unit 230 of the HMD 200 is constituted by, for example, a ROM and a RAM, stores various programs and data, and is used as a work area and a temporary data storage area when executing various programs. The control unit 210 of the HMD 200 is constituted by, for example, a CPU and controls the operation of each unit of the HMD 200 by executing a computer program read from the storage unit 230.

### D-2. Examinee-Characteristic Measurement Process

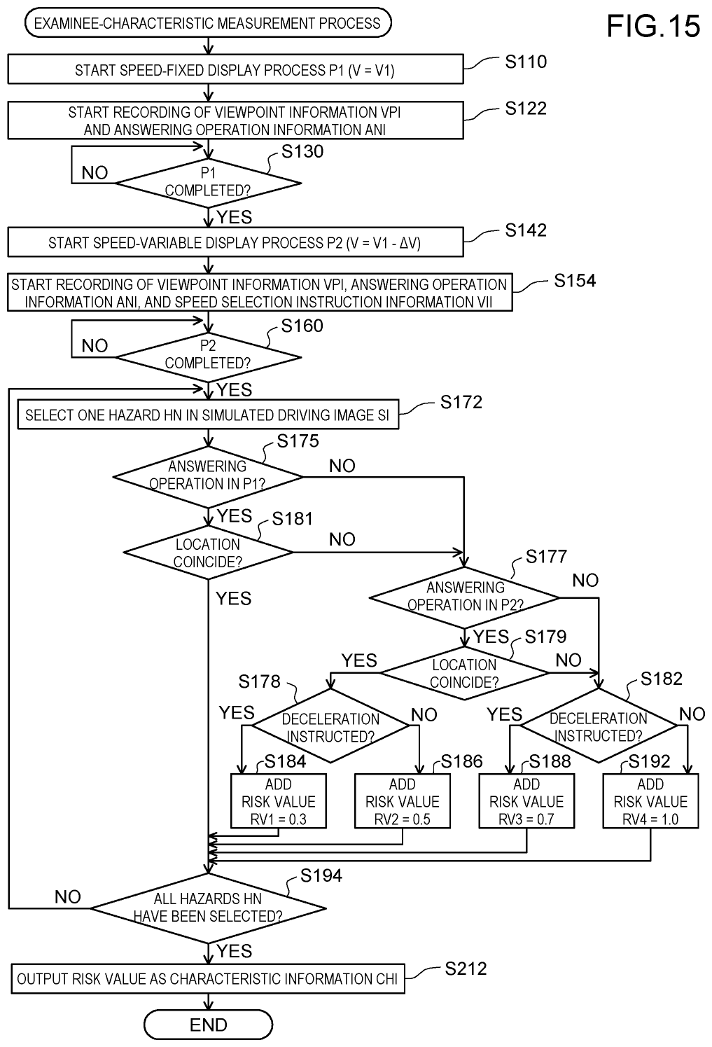

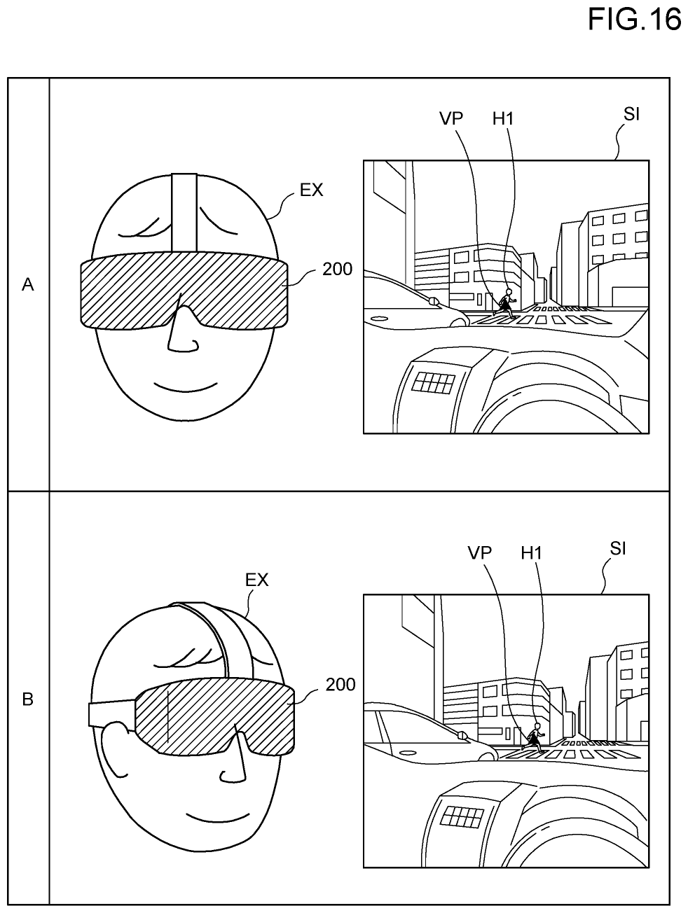

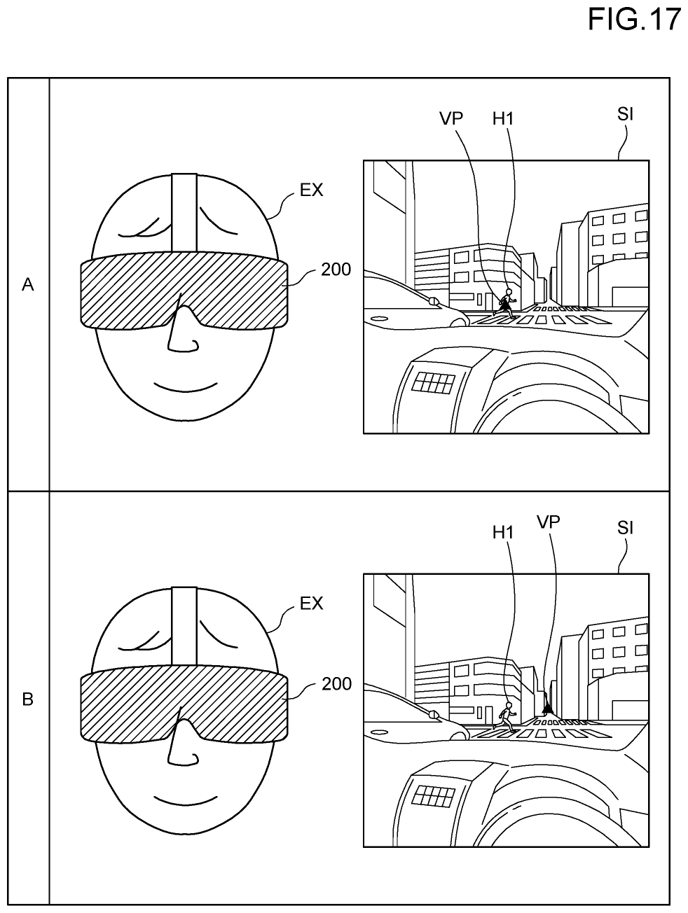

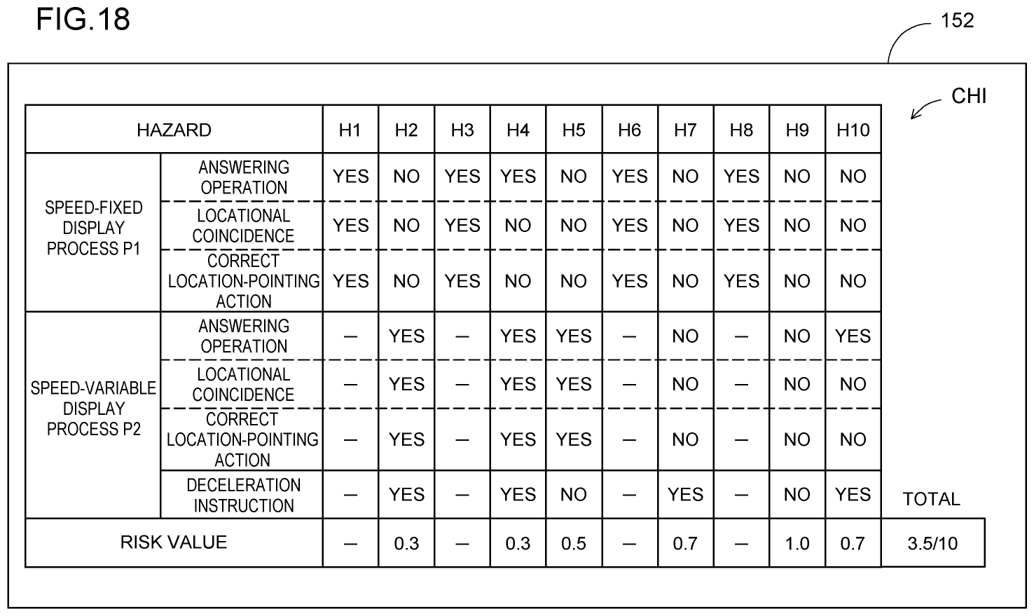

Next, the examinee-characteristic measurement process executed by the information processing system 10 of the fourth embodiment will be described. FIG. 15 is a flowchart illustrating the contents of an examinee-characteristic measurement process in the fourth embodiment. FIGS. 16 and 17 are explanatory diagrams schematically illustrating a state of the examinee EX during the examinee-characteristic measurement process in the fourth embodiment and the simulated driving image SI viewed by the examinee EX. FIG. 18 is an explanatory diagram illustrating an example of a state in which characteristic information CHI indicating the result of the examinee-characteristic measurement process in the fourth embodiment is displayed on the display unit 152.

In the examinee-characteristic measurement process of the fourth embodiment, a hazard recognition test is performed in the same way as in the examinee-characteristic measurement process of the second embodiment. However, in this embodiment, during the hazard recognition test, the examinee EX is instructed to respond by operating the operation input unit 158 (e.g., left mouse click operation) when he/she recognizes something that he/she considers to be the hazard Hn. As in the second embodiment, during the speed-variable display process P2, the examinee EX is instructed to operate the operation input unit 158 (e.g., right-click operation of the mouse) as a speed selection instruction VI (deceleration instruction) to decrease the driving speed of the car when he/she wishes to decrease the driving speed.

For example, in a state in which the examinee EX wears the HMD 200, the examinee-characteristic measurement process is started in response to an instruction for starting the process input by an administrator via the operation input unit 158 of the information processing device 100. When the examinee-characteristic measurement process is started, the speed-fixed display process P1 is started (S110) in the same way as the examinee-characteristic measurement process in the second embodiment. However, since the HMD 200 is used in the fourth embodiment, the display control unit 112 of the information processing device 100 causes the HMD 200 to start displaying the simulated driving image SI. Specifically, the display control unit 112 supplies the moving image data MID stored in the storage unit 130 to the HMD 200 and causes the right-eye display executing unit 251 and the left-eye display executing unit 252 of the HMD 200 to display the right eye image SIr and the left eye image SIl constituting the simulated driving image SI, respectively.

It should be noted that the information processing system 10 of the present embodiment has a so-called head tracking function and changes the simulated driving image SI viewed by the examinee EX according to the movement of the head of the examinee EX. That is, the head information receiving unit 111 of the information processing device 100 receives head information specifying the head movement of the examinee EX detected by the head movement detecting unit 255 of the HMD 200 from the HMD 200, and the display control unit 112 of the information processing device 100 selects the moving image data MID supplied to the right-eye display executing unit 251 and the left-eye display executing unit 252 of the HMD 200 according to the head movement of the examinee EX specified by the received head information. Thus, the examinee EX views the simulated driving image SI which naturally changes according to the movement of his/her own head. For example, when the examinee EX changes the direction of the head from a state where the examinee EX faces the front and sees an image of a scene as shown in Column A of FIG. 16 to the left as shown in Column B of FIG. 16, the image viewed by the examinee EX changes naturally to an image of a scene shifted to the left from the previous scene. Thus, the examinee EX is placed in an environment very close to an actual driving environment in terms of vision. The selection of the moving image data MID supplied to the right-eye display executing unit 251 and the left-eye display executing unit 252 may be executed by the control unit 210 of the HMD 200.

Simultaneously with the start of the speed-fixed display process P1, the viewpoint information receiving unit 113 of the information processing device 100 starts a process of receiving, from the HMD 200, viewpoint information VPI for specifying the location of the viewpoint VP of the examinee EX on the simulated driving image SI specified by the line-of-sight detecting unit 253 of the HMD 200 (S122). At the same time, the answering operation receiving unit 116 of the information processing device 100 starts accepting answering operations (operations of the operation input unit 158) by the examinee EX and starts recording the answering operation information ANI indicating the history of the operations. The received and updated viewpoint information VPI and answering operation information ANI are stored in the storage unit 130.

The viewpoint information VPI is information for specifying the location (coordinates) of the viewpoint VP of the examinee EX at each time point in the simulated driving image SI. By referring to the viewpoint information VPI, it is possible to grasp where the examinee EX is gazing at in each scene of the simulated driving image SI. The answering operation information ANI is information that identifies the time when the examinee EX responded (time point in the simulated driving image SI). By referring to the answering operation information ANI, it is possible to know at what time (i.e., in what scene) in the simulated driving image SI the examinee EX recognized something that he/she considers to be the hazard Hn. Therefore, by referring to the viewpoint information VPI and the answering operation information ANI, it is possible to know in which scene and at which location in the simulated driving image SI the examinee EX recognized the hazard Hn. Thus, the viewpoint information receiving unit 113 and the answering operation receiving unit 116 function as location-pointing action accepting unit that accepts location-pointing action performed by the examinee EX to point to a specific location in the simulated driving image SI.

It should be noted that, in FIGS. 13, 16, and 17, for example, a mark indicating the viewpoint VP is drawn over the simulated driving image SI for convenience of explanation, but in this embodiment, the mark indicating the viewpoint VP is not actually displayed as an image so that the examinee EX under test is not conscious of the location of his/her own viewpoint VP. However, a mark indicating the viewpoint VP may be displayed (may be visible to the examinee EX).

The display control unit 112 of the information processing device 100 monitors whether the speed-fixed display process P1 is completed (S130), and if it is determined that the speed-fixed display process P1 is completed (S130: YES), the process proceeds to S142. In S142, the speed-variable display process P2 is started, as with the examinee-characteristic measurement process in the second embodiment. In the speed-variable display process P2, as with the examinee-characteristic measurement process of the second embodiment, the display control unit 112 causes the simulated driving image SI to be displayed in such a manner that the driving speed decreases from the reference speed V1 to a lower speed (V1−ΔV) in response to a deceleration instruction received from the examinee EX by the speed selection instruction receiving unit 117. However, in the fourth embodiment, as in the speed-fixed display process P1, the display control unit 112 supplies the moving image data MID to the HMD 200 and causes the right-eye display executing unit 251 and the left-eye display executing unit 252 of the HMD 200 to display the right eye image SIr and the left eye image SIl constituting the simulated driving image SI, respectively.

When the speed-variable display process P2 is started, as in the speed-fixed display process P1 described above, the viewpoint information VPI and the answering operation information ANI are started to be recorded (S154), and at the same time, the speed selection instruction receiving unit 117 starts receiving deceleration instructions from examinee EX and starts recording speed selection instruction information VII, which indicates the history of the deceleration instructions. The received and updated viewpoint information VPI, answering operation information ANI, and speed selection instruction information VII are stored in the storage unit 130.

The display control unit 112 of the information processing device 100 monitors whether the speed-variable display process P2 is completed (S160), and if it is determined that the speed-variable display process P2 is completed (S160: YES), the process proceeds to S172.

When the speed-fixed display process P1 and the speed-variable display process P2 are completed, as with the examinee-characteristic measurement process in the second embodiment, the characteristic measurement unit 118 of the information processing device 100 starts the characteristic measurement for the examinee EX during driving (S172 to S194). However, in the fourth embodiment, the determination as to whether examinee EX correctly recognized each hazard Hn during the speed-fixed display process P1 and speed-variable display process P2 (processes corresponding to S174 and S176 of FIG. 9 in the second embodiment) is performed using the viewpoint information VPI and the answering operation information ANI.

In other words, the characteristic measurement unit 118 refers to the correct answer information RAI and the answering operation information ANI to determine whether there was an answering operation by the examinee EX at a specific time when the scene including hazard Hn is displayed (S175, S177) and also refers to the correct answer information RAI and the viewpoint information VPI to determine whether the location of hazard Hn matches the location of the examinee EX's viewpoint VP at the time when the scene including the hazard Hn is displayed (S181, S179), and determines that the examinee EX correctly recognized the hazard Hn when there is an answering operation by the examinee EX (S175, S177: YES) and the location of the examinee EX's viewpoint VP matches the location of hazard Hn (S181, S179: YES), and determines that the examinee EX did not correctly recognize the hazard Hn in the other cases.

For example, in the example of the characteristic measurement result shown in FIG. 18, for the scene including each of hazards H1, H3, H6, and H8 in the speed-fixed display process P1, there is an answer by the examinee EX (answering operation: YES) and the location of the hazard Hn coincided with the location of the viewpoint VP of the examinee EX (locational coincidence: YES) it is determined that the examinee EX could correctly recognize the hazard (correct location-pointing action: YES). On the other hand, for the scene including hazards H2, H5, H7, H9, and H10 in the speed-fixed display process P1, since there was no answer by the examinee EX (answering operation: NO), it is determined that examinee EX failed to recognize the hazard correctly (correct location-pointing action: NO). For the scene including hazard H4 in the speed-fixed display process P1, although the examinee EX answered (answering operation: YES), the location of the hazard Hn and the location of the viewpoint VP of the examinee EX did not match (locational coincidence: NO) so that it is determined that examinee EX failed to correctly recognize the hazard (correct location-pointing action: NO).

In this specification, the coincidence between the location of the hazard Hn and the location of the viewpoint VP of the examinee EX means that the degree of coincidence between the two locations is greater than a predetermined threshold. In other words, the characteristic measurement unit 118 determines that the location of hazard Hn and the location of the examinee EX's viewpoint VP coincides when the ratio of the length of time that the location of the viewpoint VP of the examinee EX coincides with the location (region) of hazard Hn to the length of time that the scene including hazard Hn is displayed (i.e., the degree of coincidence between the location of the hazard Hn and the location of viewpoint VP of the examinee EX) is a predetermined threshold or more. Column A of FIG. 17 shows an example where the location of the hazard Hn and the location of the examinee EX's viewpoint VP coincide and Column B of FIG. 17 shows an example where the location of the hazard Hn and the location of the examinee EX's viewpoint VP do not coincide.

When the processes (S172 to S194) for all hazards Hn are completed (S194: YES), the characteristic measurement unit 118 generates the characteristic information CHI representing the results of the characteristic measurement and outputs the characteristic information CHI (S212). For example, the characteristic measurement unit 118 displays the contents of the characteristic information CHI on the display unit 152, as shown in FIG. 18. This completes the examinee-characteristic measurement process for measuring the characteristics of the examinee EX when the examinee EX drives a car and moves on a road.

As described above, in the information processing system 10 of the fourth embodiment as well, the display control unit 112 executes the speed-variable display process P2 in which the simulated driving image SI is displayed in such a manner that the driving speed is set according to the speed selection instruction VI (deceleration instruction) received by the speed selection instruction receiving unit 117, and the characteristic measurement unit 118 measures the characteristics of the examinee EX based on the content of the speed selection instruction VI in addition to the indication results correlating with whether the location of the hazard Hn is correctly pointed to at the specific time when the scene including the hazard Hn of the simulated driving image SI is displayed during the speed-variable display process P2. Therefore, the information processing system 10 of the fourth embodiment, as with the information processing device 300 of the second embodiment, can perform the characteristic measurement for the examinee EX while considering the action of the examinee EX to select the driving speed of the car, thereby improving the appropriateness of the characteristic measurement for the examinee EX.

### E. Modifications

The technology disclosed herein is not limited to the embodiments described above and can be modified in various forms without departing from the scope of the invention, such as the following modifications.

The configuration of the information processing device 300 or the information processing system 10 in the above embodiments is merely an example and can be modified in various ways. For example, in the first through third embodiments above, a tablet-type terminal is used as the information processing device 300, but other types of computers (e.g., smartphones, PCs, or the like) may be used as the information processing device 300. Further, in the above fourth embodiment, a PC is used as the information processing device 100 constituting the information processing system 10, but other types of computers (e.g., smartphones, tablet terminals, or the like) may be used as the information processing device 100. Further, in the above fourth embodiment, the HMD 200 is used as an image display device constituting the information processing system 10, but other types of image display devices (e.g., liquid crystal displays, projectors, or the like) may be used as the image display device. It should be noted that when a device other than the HMD 200 (image display device not mounted on head) is used as the image display device, a sensor for detecting the direction of the line-of-sight of the examinee EX and a sensor for detecting the head movement of the examinee EX may be used separately from the image display device to detect the direction of the line-of-sight of the examinee EX and the head movement of the examinee EX. In the above fourth embodiment, the information processing device 100 and the image processing device constituting the information processing system 10 may be a single device. For example, the information processing system 10 may be composed of the HMD 200 provided with the functions of the information processing device 100 of the above embodiments.

The contents of the examinee-characteristic measurement process in the above embodiments are merely examples and can be modified in various ways. For example, in the second embodiment above, the speed selection instruction VI (deceleration instruction) by the examinee EX to decrease the driving speed is conducted by a predetermined operation using the operation input unit 158 or by touching the deceleration button BB displayed on the display unit 352, but this deceleration instruction can be implemented by other means (e.g., operation of a device, such as a pedal, voice operation, gesture operation, or the like).

Furthermore, in the first embodiment above, the choices of driving speed in the speed-variable display process P2 include a speed higher than the reference speed V1, which is the driving speed in the speed-fixed display process P1, a speed the same as the reference speed V1, and a speed lower than the reference speed V1, but the choices may not include at least one of these. In the speed-variable display process P2 of the above second embodiment, only the speed selection instruction VI to decrease the driving speed from the reference speed V1 (deceleration instruction) is possible, but the speed selection instruction VI to increase the driving speed from the reference speed V1 (acceleration instruction) may be executable. In the above embodiment, a scene including a speed limit sign may be provided in the simulated driving image SI, and if a speed selection instruction VI is executed to select a speed exceeding the speed limit indicated on the sign, even though the examinee EX recognized the speed limit sign (touched the sign on the screen) the examinee may be found to have a poor attitude towards complying with legal and regulatory requirements in addition to having a high risk-taking tendency. In the above embodiment, when outputting the characteristic information CHI indicating the characteristics of the examinee EX, an explanation of the importance of selecting a speed according to the situation and future advice according to aging changes may be provided. In the above embodiment, the characteristic measurement results of the examinee in question may be stored, and determination and feedback in comparison with the past results may be provided (e.g., “Last year, the indication rate was high without reducing the speed, but this time the indication rate has decreased from last year, so reducing the speed is recommended). In the above embodiment, past characteristic measurement results for a number of examinees may be stored, and a relative evaluation of the characteristic measurement result for the current examinee (e.g., the position of the current examinee relative to the average of all examinees) may be output.

The stages and determination criteria set for each characteristic (hazard-perception ability, self-evaluation ability, self-control ability, and risk-taking tendency) of the examinee EX in the first embodiment above are only examples and can be changed in various ways. In the first embodiment above, hazard-perception ability, self-evaluation ability, self-control ability, and risk-taking tendency are used as the characteristics of the examinee EX, but at least one of these may not be used as the characteristics of the examinee EX, or other characteristics may be used in addition to these characteristics.

In the above first embodiment, the self-evaluation SE of the indication rate evaluated by the examinee EX is received for the speed-fixed display process P1, and the self-evaluation SE is used to measure the characteristics of the examinee EX, but the self-evaluation SE of the indication rate evaluated by the examinee EX may be received with respect to the speed-variable display process P2 instead of or together with the speed-fixed display process P1, and such self-evaluation SE may be used to measure the characteristics of the examinee EX.

In the examinee-characteristic measurement process in the above fourth embodiment, when the examinee EX recognizes each hazard Hn in the simulated driving image SI during the hazard recognition test, the examinee EX answers by operating the operation input unit 158, but the method of answering is not limited to the operation of the operation input unit 158 and may be another method. For example, without relying on the operation of the operation input unit 158, it may be determined that the examinee EX recognized the hazard Hn when the ratio of the length of time during which the location of the viewpoint VP of the examinee EX coincides with the location (region) of the hazard Hn to the length of time during which the scene including the hazard Hn is displayed (i.e., the degree of coincidence between the location of the hazard Hn and the location of the viewpoint VP of the examinee EX) is a predetermined threshold or more.

In the first, second, and fourth embodiments above, the speed-variable display process P2 is performed after the speed-fixed display process P1, but conversely, the speed-fixed display process P1 may be performed after the speed-variable display process P2. In the above fourth embodiment, the speed-fixed display process P1 and the speed-variable display process P2 are executed, but as in the above third embodiment, only the speed-variable display process P2 may be executed. In each of the above embodiments, the hazard recognition test may be repeatedly executed until the examinee EX meets predetermined criteria (e.g., until the indication rate exceeds a predetermined threshold). In each of the above embodiments, scores of the hazard recognition test may be used instead of the indication rate or the like. A score of the hazard recognition test is calculated, e.g., by using the speed selected by the speed selection instruction VI in addition to the indication rate and by a function of them (e.g., addition or multiplication of the indication rate and speed).

In the examinee-characteristic measurement process in each of the above embodiments, the simulated driving image SI includes scenes including hazards Hn, and the characteristics of the examinee EX during driving are measured by determining whether examinee EX correctly recognized each hazard Hn; however, the target objects to be recognized by the examinee EX are not limited to hazards Hn but can be anything that is not a hazard but influences the characteristics of the examinee EX during driving, such as traffic signals and road signs. The target object may also be something that predicts future changes, such as a blinking green light of a pedestrian signal. In other words, in the examinee-characteristic measurement process in each of the above embodiments, a target object recognition test may be performed to determine whether such a target object can be recognized, and the target object perception ability as a characteristic of the examinee EX may be measured based on the test results.

In each of the above embodiments, the simulated driving image SI may include a target object representing actual risk and a target object representing potential risk, and the characteristic measurement unit may evaluate the risk caused by the visual field defect based on the result of the indication result at the specific time when a scene including a target object representing actual risk is displayed and the risk caused by cognitive function decline based on the indication results at the specific time when a scene including the target object representing potential risk is displayed. This improves the appropriateness of the evaluation of the risk caused by the visual field defect and the evaluation of the risk caused by cognitive function decline.

In each of the above embodiments, the characteristic information CHI is output by being displayed on the display units 152, 352, but the characteristic information CHI may be output in other forms, such as output by voice or by printing using a printing device. In the above embodiment, the content of the output characteristic information CHI is merely an example and can be modified in various ways. For example, the characteristic information CHI may include the result of the indication rate, or it may include only the final total risk value (e.g., 3.5/10 points) without including the contents indicating the suitability of recognition for each hazard Hn.

Although each of the above embodiments involves measuring the characteristics of the examinee EX when the examinee EX drives a car and moves on a road, the technology disclosed herein is equally applicable to measuring the characteristics of the examinee EX when the examinee EX moves by other means (e.g., driving other types of vehicles (such as bicycles), or on foot).

Moreover, in each of the above-described embodiments, a part of the configuration implemented by hardware may be replaced with software, and on the contrary, a part of the configuration implemented by software may be replaced with hardware.

### REFERENCE SIGNS LIST

10: information processing system, 12: cable, 100: information processing device, 110: control unit, 111: head information receiving unit, 112: display control unit, 113: viewpoint information receiving unit, 116: answering operation receiving unit, 117: speed selection instruction receiving unit, 118: characteristic measurement unit, 130: storage unit, 152: display unit, 158: operation input unit, 159: interface unit, 190: bus, 200: HMD, 210: control unit, 230: storage unit, 251: right-eye display executing unit, 252: left-eye display executing unit, 253: line-of-sight detecting unit, 254: headphone, 255: head movement detecting unit, 258: operation input unit, 259: interface unit, 290: bus, 300: information processing device, 310: control unit, 312: display control unit, 315: self-evaluation receiving unit, 316: location-pointing action accepting unit, 317: speed selection instruction receiving unit, 318: characteristic measurement unit, 330: storage unit, 352: display unit, 358: operation input unit, 359: interface unit, 390: bus

1. An information processing device for measuring characteristics of an examinee when the examinee moves, comprising:
a speed selection instruction receiving unit that receives a speed selection instruction for selecting a moving speed by the examinee;
a display control unit that executes a speed-variable display process to display on an image display unit a simulated moving image, which is a moving image simulating the field of vision of a human moving along a predetermined course and including at least one target object in such a manner that the moving speed is set in accordance with the speed selection instruction;
a location-pointing action accepting unit that accepts a location-pointing action performed by the examinee to point to a specific location in the simulated moving image; and
a characteristic measurement unit that measures the characteristics based on the indication results correlating with whether the location-pointing action correctly pointed to the location of the target object at a specific time when the scene including each target object in the simulated moving image is displayed in the speed-variable display process and on the content of the speed selection instruction, and outputs characteristic information representing the measurement results of the characteristics.

2. The information processing device according to claim 1, wherein
the display control unit further executes a speed-fixed display process to display the simulated moving image on the image display unit in a manner in which the moving speed is fixed to a preset reference speed, and
the characteristic measurement unit measures the characteristics based on the indication results in the speed-fixed display process.

3. The information processing device according to claim 2, wherein
the display control unit executes the speed-variable display process after execution of the speed-fixed display process, and
the speed selection instruction receiving unit receives the speed selection instruction after execution of the speed-fixed display process and before execution of the speed-variable display process.

4. The information processing device according to claim 1, wherein
the characteristics include the target object perception ability and the self-control ability of the examinee.

5. The information processing device according to claim 1, wherein
the characteristic includes the self-evaluation ability of the examinee,
the information processing device further comprises a self-evaluation receiving unit that receives a self-evaluation of the indication results evaluated by the examinee, and
the characteristic measurement unit measures the characteristic based on the self-evaluation.

6. The information processing device according to claim 5, wherein
the characteristic measurement unit determines the self-evaluation ability of the examinee based on the difference between the self-evaluation and the actual indication results.

7. The information processing device according to claim 2, wherein
the speed selection instruction receiving unit receives the speed selection instruction during the speed-variable display process, and
the display control unit changes the moving speed in accordance with the speed selection instruction during the speed-variable display process.

8. The information processing device according to claim 7, wherein
the characteristic includes a risk of the examinee while moving, and
the characteristic measurement unit determines that in a case where there is no location-pointing action correctly pointing to the location of the target object at the specific time during the speed-variable display process, the risk is lower when there is a speed selection instruction to reduce the moving speed than when there is no speed selection instruction to reduce the moving speed.

9. The information processing device according to claim 7, wherein
the characteristic includes a risk of the examinee while moving, and
the characteristic measurement unit determines that in a case where there is no location-pointing action correctly pointing to the location of the target object at the specific time during the speed-fixed display process and there is no location-pointing action correctly pointing to the location of the target object at the specific time during the speed-variable display process, the risk is lower when there is a speed selection instruction to reduce the moving speed than when there is no speed selection instruction to reduce the moving speed.

10. The information processing device according to claim 9, wherein
the characteristic measurement unit determines that in a case where there is no location-pointing action correctly pointing to the location of the target object at the specific time during the speed-fixed display process and there is a location-pointing action correctly pointing to the location of the target object at the specific time during the speed-variable display process, the risk is lower when there is a speed selection instruction to reduce the moving speed than when there is no speed selection instruction to reduce the moving speed.

11. The information processing device according to claim 7, wherein
the display control unit executes the speed-variable display process after execution of the speed-fixed display process.

12. The information processing device according to claim 1, wherein
the simulated moving image includes the target object representing actual risk and the target object representing potential risk,
the characteristics include the risk of the examinee while moving, and
the characteristic measurement unit measures the risk due to a visual field defect based on the indication result at the specific time when a scene including the target object representing actual risk is displayed, and the risk due to cognitive function decline based on the indication result at the specific time when a scene including the target object representing potential risk is displayed.

13. The information processing device according to claim 1, wherein
the simulated moving image is a moving image simulating the field of vision of a human driving a vehicle and moving on the course.

14. The information processing device according to claim 1, further comprising
the image display unit.

15. An information processing system comprising: the information processing device according to claim 1; and
an image display device as the image display unit.

16. An information processing method for measuring characteristics of an examinee when the examinee moves, comprising:
a step of receiving a speed selection instruction for selecting a moving speed by the examinee;
a step of executing a speed-variable display process to display on an image display unit a simulated moving image, which is a moving image simulating the field of vision of a human moving along a predetermined course and including at least one target object in such a manner that the moving speed is set in accordance with the speed selection instruction;
a step of accepting a location-pointing action performed by the examinee to point to a specific location in the simulated moving image; and
a step of measuring the characteristics based on the indication results correlating with whether the location-pointing action correctly pointed to the location of the target object at a specific time when the scene including each target object in the simulated moving image is displayed in the speed-variable display process and on the content of the speed selection instruction, and outputting characteristic information representing the measurement results of the characteristics.

17. A computer program for measuring characteristics of an examinee when the examinee moves, causing a computer to execute:
a process of receiving a speed selection instruction for selecting a moving speed by the examinee;
a speed-variable display process to display on an image display unit a simulated moving image, which is a moving image simulating the field of vision of a human moving along a predetermined course and including at least one target object in such a manner that the moving speed is set in accordance with the speed selection instruction;
a process of accepting a location-pointing action performed by the examinee to point to a specific location in the simulated moving image; and
a process of measuring the characteristics based on the indication results correlating with whether the location-pointing action correctly pointed to the location of the target object at a specific time when the scene including each target object in the simulated moving image is displayed in the speed-variable display process and on the content of the speed selection instruction, and outputting characteristic information representing the measurement results of the characteristics.
